(19) United States
(12) Patent Application Publication
(10) Pub. No.: US 2024/0207432 A1
(43) Pub. Date: Jun. 27, 2024

(54) ANTIBODIES BINDING TROP2 AND USES THEREOF

(71) Applicant: BIOSION INC., Nanjing, Jiangsu (CN)

(72) Inventor: Mingjiu CHEN, Nanjing (CN)

(21) Appl. No.: 18/555,570

(22) PCT Filed: Apr. 21, 2021
(86) PCT No.: PCT/CN2022/088165

§ 371 Date: Oct. 16, 2023

Related U.S. Application Data
(60) Provisional application No. 63/178,741, filed on Apr. 23, 2021.

(51) Int. Cl. A61K 47/68; A61K 51/10; C07K 16/30

(52) U.S. Cl. CPC A61K 47/6851; A61K 47/6811; A61K 51/1045; C07K 16/30; C07K 2317/20; C07K 2317/24; C07K 2317/92

(57) ABSTRACT

Provided is an isolated monoclonal antibody that specifically binds human TROP2, or the antigen-binding portion thereof. A nucleic acid molecule encoding the antibody or the antigen-binding portion thereof, an expression vector, a host cell and a method for expressing the antibody or the antigen-binding portion thereof are also provided. Further provided are a bispecific molecule, an immunoconjugate, a chimeric antigen receptor, an oncolytic virus and a pharmaceutical composition comprising the antibody or the antigen-binding portion thereof, as well as a treatment method using an anti-TROP2 antibody or the antigen-binding portion thereof.

### RELATED APPLICATIONS AND INCORPORATION BY REFERENCE

This application claims priority to U.S. provisional patent application Ser. No. 63/178,741 filed on Apr. 23, 2021.

The foregoing application, all documents cited therein (“appln cited documents”), all documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### FIELD OF THE INVENTION

The present disclosure relates generally to an isolated monoclonal antibody, particularly a mouse, chimeric or humanized monoclonal antibody, or an antigen-binding portion thereof, that binds to human TROP2, with high affinity and functionality. A nucleic acid molecule encoding the antibody or the antigen-binding portion thereof, an expression vector, a host cell and a method for expressing the antibody or the antigen-binding portion thereof are also provided. The present disclosure further provides a bispecific molecule, an immunoconjugate, a chimeric antigen receptor, an oncolytic virus, and a pharmaceutical composition which may comprise the antibody or the antigen-binding portion thereof, as well as a treatment method using the same.

### BACKGROUND OF THE INVENTION

TROP2 is a transmembrane glycoprotein that is also known as epithelial glycoprotein-1 (EGP-1), membrane component surface marker-1 (M1S1), tumor-associated calcium signal transducer-2 (TACSTD2) and gastrointestinal antigen 733-1 (GA733-1). Each TROP2 molecule is composed of a hydrophobic precursor peptide, an extracellular domain, a transmembrane domain and a cytoplasmic tail. The cytoplasmic tail contains a highly conserved phosphatidylinositol 4, 5-bisphosphate (PIP2) binding sequence and a serine phosphorylation site at position 303 (Zaman S et al., (2019) Onco Targets Ther. 12:1781-1790). The binding partners of TROP2 include IFG-1, Claudin-1, Claudin-7, cyclin D1 and PKC (Shvartsur A et al., (2015) Genes Cancer. 6(3-4):84-105).

TROP2 is expressed at low levels in normal tissues playing a role in e.g., embryonic organ development and fetal growth, while upregulated TROP2 expression has been found in all cancer types independent of the baseline TROP2 levels in normal counterparts (Mustata R C et al., (2013) Cell Reports. 5(2):421-432; Guerra E et al., (2012) PLoS ONE. 7(11): e49302; Trerotola M et al., (2013) Oncogene. 32(2): 222-233). Studies have shown several transcription factors on which TROP2 expression depends are correlated with cancer development, such as TP63/fP53L and Wilm's tumor 1 (WT1), and TROP2 is demonstrated to be involved in many cell signaling pathways associated with tumorigenesis. For example, TROP2 signaling regulates cell self-renewal and proliferation via β-catenin signaling, and thus promotes stem cell-like properties of cancer cells (Stoyanova T et al., (2012) Genes Dev. 26(20):2271-2285). TROP2 overexpression promotes tumor invasion in cervical, ovarian, colon and thyroid cancers, and TROP2 knock-down decreases cancer cell invasion (Guan H et al., (2017) BMC Cancer. 17(1):486; Liu T et al., (2013) PLoS One. 8(9):e75864: Wu B et al., (2017) Exp Ther Med. 14(3):1947-1952; Zhao P et al., (2018) Oncol Lett. 15(3):3820-3827). Recently, TROP2 signaling has been further found to modulate signaling for cell migration. For instance, it was reported that TROP2 regulates 01 integrin functions to promote prostate cancer metastasis (Trerotola M et al., (2013) Cancer Res. 73(10):3155-3167).

High TROP2 expression has been clinically correlated with poor prognosis in e.g., hilar cholangiocarcinoma, cervical cancer, and gastric cancer. In a meta-analysis including 2,569 patients, TROP2 expression increase was statistically linked to poor overall and disease-free survival outcomes in several solid tumors (Fong D et al., (2008) Br J Cancer. 99(8):1290-1295; Ning S et al., (2013) J Gastrointest Surg. 17(2):360-368; Liu T et al., (2013) PLoS One. 8(9):e75864; Zhao W et al., (2016) Oncotarget. 7(5):6136-6145; Zeng P et al., (2016) Sci Rep. 6:33658). TROP2's role as a tumor marker is being tested in a certain clinical trial.

Because of its structure characteristic and correlation with cancer, TROP2 has become an attractive therapeutic target. Several anti-TROP2 antibodies were prepared, some of which were found to inhibit breast cancer progression and induce apoptosis in xenograft mouse model (Lin H et al., (2014) Int J Cancer. 134(5):1239-1249). However, none showed therapeutic value as a naked antibody, probably due to their high internalization rates, until Pr1E11 was identified by IKEDA et al., in 2015 with higher binding affinity and lower internalization activity (Ikeda M et al., (2015) Biochem Biophys Res Commun. 458(4):877-82). Pr1E11 was determined in a later study to induce potent antibody-dependent cytotoxicity in vivo, which was presumed to be high cell surface retention related (Ikeda M et al., (2016) Anticancer Res. 36(11):5937-5944). Currently, most TROP2 targeted therapeutics that are under pre-clinical and clinical trials are antibody-drug conjugates (ADCs), including DS-1062a, IMMU-132 and PF-06664178, with some encouraging outcomes obtained till now in solid cancer treatment with limited toxicity (Zaman S et al., (2019) supra). The novel TROP2-directed antibody-drug conjugate (ADC), datopotamab deruxtecan (Dato-DXd, DS-1062a), with a potent DNA topoisomerase I inhibitor (DXd) was developed, and its antitumor activity and safety profiles in preclinical models was evaluated (Daisuke Okajima et al., Mol Cancer Ther, 2021 December; 20(12): 2329-2340).

There is a need for additional anti-TROP2 antibodies with low internalization activity to be used as naked antibodies or with high internalization activity for ADC preparation.

Citation or identification of any document in this application is not an admission that such document is available as prior art to the present invention.

### SUMMARY OF THE INVENTION

The present disclosure provides an isolated monoclonal antibody, for example, a mouse, chimeric or humanized monoclonal antibody, or an antigen-binding portion thereof, that binds to TROP2 (e.g., human TROP2) and has comparable, if not higher, binding affinity/capability to human and/or monkey TROP2, and higher or lower internalization activity, as compared to prior art anti-TROP2 antibodies such as sacituzumab (the antibody part of IMMU-132).

The antibody or antigen-binding portion of the disclosure can be used for a variety of applications, including detection of TROP2 proteins in vitro, and treatment of TROP2 related diseases, such as cancers.

Accordingly, in one aspect, the disclosure pertains to an isolated monoclonal antibody (e.g., a mouse, chimeric or humanized antibody), or an antigen-binding portion thereof, that binds TROP2, comprising (i) a heavy chain variable region that may comprise a VH CDR1 region, a VH CDR2 region and a VH CDR3 region, wherein the VH CDR1 region, the VH CDR2 region and the VH CDR3 region may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 1, 2 and 3, respectively; (2) SEQ ID NOs: 7, 8 and 3, respectively; (3) SEQ ID NOs: 12, 13 and 14, respectively; (4) SEQ ID NOs: 18, 19 and 20, respectively; (5) SEQ ID NOs: 24, 25 and 26, respectively; (6) SEQ ID NOs: 30, 31 and 32, respectively; or (7) SEQ ID NOs: 35, 36 and 37, respectively; and/or (ii) a light chain variable region that may comprise a VL CDR1 region, a VL CDR2 region and a VL CDR3 region, wherein the VL CDR1 region, the VL CDR2 region and the VL CDR3 region may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 4, 5 and 6, respectively; (2) SEQ ID NOs: 9, 10 and 11, respectively; (3) SEQ ID NOs: 15, 16 and 17, respectively; (4) SEQ ID NOs: 21, 22 and 23, respectively; (5) SEQ ID NOs: 27, 28 and 29, respectively; (6) SEQ ID NOs: 33, 34 and 29, respectively; or (7) SEQ ID NOs: 38, 39 and 40, respectively.

The isolated monoclonal antibody, or the antigen-binding portion thereof, of the present disclosure may comprise a heavy chain variable region having a VH CDR1 region, a VH CDR2 region and a VH CDR3 region, and a light chain variable region having a VL CDR1 region, a VL CDR2 region and a VL CDR3 region, wherein the VH CDR1, VH CDR2, VH CDR3, VL CDR1, VL CDR2 and VL CDR3 may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 1, 2, 3, 4, 5 and 6, respectively; (2) SEQ ID NOs: 7, 8, 3, 9, 10 and 11, respectively; (3) SEQ ID NOs: 12, 13, 14, 15, 16 and 17, respectively; (4) SEQ ID NOs: 18, 19, 20, 21, 22 and 23, respectively; (5) SEQ ID NOs: 24, 25, 26, 27, 28 and 29, respectively; (6) SEQ ID NOs: 30, 31, 32, 33, 34 and 29, respectively; or (7) SEQ ID NOs: 35, 36, 37, 38, 39 and 40, respectively.

The isolated monoclonal antibody, or the antigen-binding portion thereof, of the present disclosure may comprise a heavy chain variable region that may comprise an amino acid sequence having at least 85%, 86%, 87%, 88%, 89%, 90%, 91%, 92%, 93%, 94%, 95%, 96%, 97%, 98%, 99% or 100% identity to SEQ ID NOs: 44, 45, 46 (X1=S, X2=A; X1=T, X2=A; X1=S, X2=V), 47 (X1=R, X2=R; X1=A, X2=T), 51, 53, 55, 57, 59 or 61. The amino acid sequences of SEQ ID NOs: 44 and 47 (X1=A, X2=T) may be encoded by the nucleic acid sequences of SEQ ID NOs: 41 and 42, respectively.

The isolated monoclonal antibody, or the antigen-binding portion thereof, of the present disclosure may comprise a light chain variable region that may comprise an amino acid sequence having at least 85%, 86%, 87%, 88%, 89%, 90%, 91%, 92%, 93%, 94%, 95%, 96%, 97%, 98%, 99% or 100% identity to SEQ ID NOs: 48, 49 (X1=D, X2=L, X3=V; X1=E, X2=V, X3=L), 50 (X1=Q, X2=S, X3=K; X1=G. X2=A, X3=K; X1=G, X2=S, X3=Y), 52, 54, 56, 58, 60 or 62. The amino acid sequences of SEQ ID NOs: 48 and 50 (X1=G, X2=A, X3=K) may be encoded by the nucleic acid sequences of SEQ ID NOs: 43 and 63, respectively.

The isolated monoclonal antibody, or the antigen-binding portion thereof, of the present disclosure may comprise a heavy chain variable region and a light chain variable region, the heavy chain variable region and the light chain variable region may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 44 and 48, respectively; (2) SEQ ID NOs: 45 and 49 (X1=D, X2=L, X3=V), respectively; (3) SEQ ID NOs: 46 (X1=S, X2=A) and 49 (X1=E, X2=V, X3=L), respectively; (4) SEQ ID NOs: 46 (X1=T, X2=A) and 49 (X1=E, X2=V, X3=L), respectively; (5) SEQ ID NOs: 46 (X1=S, X2=V) and 49 (X1=E, X2=V, X3=L), respectively; (6) SEQ ID NOs: 47 (X1=R, X2=R) and 49 (X1=E, X2=V, X3=L), respectively; (7) SEQ ID NOs: 47 (X1=A, X2=T) and 49 (X1=E, X2=V, X3=L), respectively; (8) SEQ ID NOs: 46 (X1=S, X2=A) and 50 (X1=Q, X2=S, X3=K), respectively; (9) SEQ ID NOs: 46 (X1=T, X2=A) and 50 (X1=Q, X2=S, X3=K), respectively; (10) SEQ ID NOs: 46 (X1=S, X2=V) and 50 (X1=Q, X2=S, X3=K), respectively; (11) SEQ ID NOs: 47 (X1=R, X2=R) and 50 (X1=Q, X2=S, X3=K), respectively; (12) SEQ ID NOs: 47 (X1=A, X2=T) and 50 (X1=Q, X2=S, X3=K), respectively; (13) SEQ ID NOs: 46 (X1=S, X2=A) and 50 (X1=G, X2=A, X3=K), respectively; (14) SEQ ID NOs: 46 (X1=T, X2=A) and 50 (X1=G, X2=A, X3=K), respectively; (15) SEQ ID NOs: 46 (X1=S, X2=V) and 50 (X1=G. X2=A, X3=K), respectively; (16) SEQ ID NOs: 47 (X1=R, X2=R) and 50 (X1=G, X2=A, X3=K), respectively; (17) SEQ ID NOs: 47 (X1=A, X2=T) and 50 (X1=G, X2=A, X3=K), respectively; (18) SEQ ID NOs: 46 (X1=S, X2=A) and 50 (X1=G, X2=S, X3=Y), respectively; (19) SEQ ID NOs: 46 (X1=T, X2=A) and 50 (X1=G, X2=S, X3=Y), respectively; (20) SEQ ID NOs: 46 (X1=S, X2=V) and 50 (X1=G, X2=S, X3=Y), respectively; (21) SEQ ID NOs: 47 (X1=R. X2=R) and 50 (X1=G, X2=S, X3=Y), respectively; (22) SEQ ID NOs: 47 (X1=A, X2=T) and 50 (X1=G, X2=S, X3=Y), respectively; (23) SEQ ID NOs: 51 and 52, respectively; (24) SEQ ID NOs: 53 and 54, respectively; (25) SEQ ID NOs: 55 and 56, respectively; (26) SEQ ID NOs: 57 and 58, respectively; (27) SEQ ID NOs: 59 and 60, respectively; or (28) SEQ ID NOs: 61 and 62, respectively.

The isolated monoclonal antibody, or the antigen-binding portion thereof, of the present disclosure may comprise a heavy chain and a light chain linked by disulfide bonds, the heavy chain may comprise a heavy chain variable region and a heavy chain constant region, the light chain may comprise a light chain variable region and a light chain constant region, wherein the C terminus of the heavy chain variable region is linked to the N terminus of the heavy chain constant region, and the C terminus of the light chain variable region is linked to the N terminus of the light chain constant region, wherein the heavy chain variable region and the light chain variable region may comprise amino acid sequences described above, and the antibody or antigen-binding portion thereof binds to TROP2. The heavy chain constant region may be a heavy chain constant region with enhanced FcR binding capability, such as human IgG1 constant region having the amino acid sequence set forth in e.g., SEQ ID NO.: 64 (X1=R, X2=E, X3=M; X1=K, X2=D, X3=L), or a functional fragment thereof. The light chain constant region may be human kappa constant region having the amino acid sequences set forth in e.g., SEQ ID NO.: 65, or a functional fragment thereof. The heavy chain constant region may also be human IgG2 or IgG4 constant region, or a functional fragment thereof, engineered to have enhanced FcR binding affinity. The amino acid sequences of SEQ ID NOs: 64 and 65 may be encoded by the nucleic acid sequences of SEQ ID NOs: 74 and 75, respectively.

The antibody of the present disclosure in certain embodiments may comprise or consist of two heavy chains and two light chains, wherein each heavy chain may comprise the heavy chain constant region, heavy chain variable region or CDR sequences mentioned above, and each light chain may comprise the light chain constant region, light chain variable region or CDR sequences mentioned above, wherein the antibody binds to TROP2. The antibody of the disclosure can be a full-length antibody, for example, of an IgG1, IgG2 or IgG4 isotype. The antibody or the antigen-binding portion thereof of the present disclosure in other embodiments may be a single chain variable fragment (scFv) antibody, or antibody fragments, such as Fab or F(ab′)₂ fragments.

The disclosure also provides a bispecific molecule that may comprise the antibody, or the antigen-binding portion thereof, of the disclosure, linked to a second functional moiety (e.g., a second antibody) having a different binding specificity than said antibody, or antigen-binding portion thereof. The disclosure also provides an immunoconjugate, such as an antibody-drug conjugate, that may comprise an antibody, or antigen-binding portion thereof, of the disclosure, linked to a therapeutic agent, such as a cytotoxin, e.g., SN-38. In another aspect, the antibody or the antigen binding portion thereof of the present disclosure can be made into part of a chimeric antigen receptor (CAR). Also provided is an immune cell that may comprise the antigen chimeric receptor, such as a T cell and a NK cell. Further provided is an oncolytic virus armed with the antibody or the antigen binding portion thereof of the present disclosure.

The antibody or antigen-binding portion thereof, the immunoconjugate, or the bispecific molecule may be radioactively labeled and used in clinical imaging to e.g., trace/detect the distribution of TROP2⁺ tumors/cancers, including distribution of metastatic TROP2⁺ tumors/cancers. The radioactive label includes, but not limited to, ³H.

The disclosure also provides a nucleic acid molecule encoding the antibody or the antigen-binding portion thereof, the bispecific molecule, the immunoconjugate or the CAR of the disclosure, as well as an expression vector that may comprise such a nucleic acid molecule and a host cell that may comprise such an expression vector. A method for preparing the anti-TROP2 antibody or the antigen-binding portion thereof, the bispecific molecule, the immunoconjugate or the CAR of the disclosure using the host cell is also provided, that may comprise steps of (i) expressing the subject molecule in the host cell and (ii) isolating the subject molecule from the host cell or its cell culture.

Also provided is a pharmaceutical composition that may comprise the antibody or the antigen-binding portion thereof, the immunoconjugate, the bispecific molecule, the oncolytic virus, the CAR or CAR-T cell, the nucleic acid molecule, the expression vector, or the host cell of the disclosure, and a pharmaceutically acceptable carrier. In certain embodiments, the pharmaceutical composition may further contain a therapeutic agent for treating a specific disease, such as an anti-cancer agent.

In yet another aspect, the disclosure provides a method for treating a disease associated with TROP2 (e.g., excessive TROP2 expression/signaling) in a subject in need thereof, which may comprise administering to the subject a therapeutically effective amount of the pharmaceutical composition of the present disclosure. The disease may be a tumor or cancer. The tumor may be a solid tumor or a non-solid tumor, including, but not limited to, breast cancer, colorectal cancer, gastric adenocarcinoma, esophageal cancer, hepatocellular carcinoma, non-small-cell lung cancer, small-cell lung cancer, ovarian epithelial cancer, prostate cancer, pancreatic ductal adenocarcinoma, head and neck cancer, squamous cell cancer, renal cell cancer, urinary bladder neoplasm, cervical cancer, endometrial cancer, follicular thyroid cancer, and glioblastoma multiforme. In certain embodiments, at least one additional anti-cancer antibody may be further administered, such as an anti-VISTA antibody, an anti-PD-1 antibody, an anti-PD-L1 antibody, an anti-LAG-3 antibody, an anti-CTLA-4 antibody, an anti-TIM3 antibody, an anti-STAT3 antibody, and/or an anti-ROR1 antibody. In certain embodiments, the subject is human.

In another aspect, the disclosure provides a method for cancer imaging in a subject in need thereof, comprising administering the subject with a radioactively labeled anti-TROP2 antibody or antigen-binding portion thereof, the immunoconjugate, or the bispecific molecule of the disclosure. The method may be used to trace/detect the distribution of a tumor or cancer with high TROP2 expression, including, but not limited to, esophageal squamous cell carcinoma, colorectal cancer, pancreatic cancer, colon cancer, papillary thyroid cancer, breast cancer, and bladder cancer. In certain embodiments, the subject is human.

Other features and advantages of the instant disclosure will be apparent from the following detailed description and examples, which should not be construed as limiting. The contents of all references, Genbank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments described, may best be understood in conjunction with the accompanying drawings.

FIGS. 1A-1B show the binding capability of mouse antibodies A1E4F7D4, A1B12D2B4E7B3, A1E11A12D1, A1F1G12A7 and A1H3C5H8E12 (A), B1G1F5A3 and C1B3B12D2 (B) to human TROP2 in a capture ELISA.

FIGS. 2A-2B show the binding capability of mouse antibodies A1E4F7D4, A1B12D2B4E7B3, A1E11A12D1, A1F1G12A7 and A1H3C5H8E12(A), B1G1F5A3 and C1B3B12D2(B) to cynomolgus TROP2 in an indirect ELISA.

FIGS. 3A-3B show the binding capability of mouse antibodies A1E4F7D4, A1B12D2B4E7B3, A1E11A12D1, A1F1G12A7 and A1H3C5H8E12 (A), B1G1F5A3 and C1B3B12D2 (B) to 293F-TROP2 cells expressing human TROP2 in a cell based binding FACS assay.

FIGS. 4A-4C show the capability of mouse antibodies A1E4F7D4, A1E11A12D1 and A1H3C5H8E12 (A). A1F1G12A7 and A1B12D2B4E7B3 (B). B1G1F5A3 and C1B3B12D2 (C) to block benchmark-human TROP2 binding in a competitive ELISA test.

FIG. 5 shows the capability of mouse antibodies A1E4F7D4, A1E11A12D1 and A1H3C5H8E12 to block mouse antibody A1E4F7D4-human TROP2 binding in a competitive ELISA test.

FIG. 6 shows the capability of mouse antibodies A1E4F7D4, A1E11A12D1 and A1H3C5H8E12 to block mouse antibody A1E11A12D1-human TROP2 binding in a competitive ELISA test.

FIG. 7 shows the capability of mouse antibodies A1E4F7D4, A1E11A12D1 and A1H3C5H8E12 to block mouse antibody A1H3C5H8E12-human TROP2 binding in a competitive ELISA test.

FIG. 8 shows the internalization-mediated cellular toxicities of mouse antibody-DTTP1170 conjugates on 293F-TROP2 cells.

FIGS. 9A-9B show the binding capability of chimeric antibodies A1E4F7D4 and C1B3B12D2 (A), and A1F1G12A7 (B) to human TROP2 in a capture ELISA.

FIGS. 10A-10B show the binding capability of chimeric antibodies A1E4F7D4 and C1B3B12D2 (A), and A1F1G12A7 (B) to cynomolgus TROP2 in an indirect ELISA.

FIGS. 11A-11B show the binding capability of chimeric antibodies A1E4F7D4 and C11B3B12D2 (A), and A1F1G12A7 (B) to 293F-TROP2 cells expressing human TROP2 in a cell based binding FACS assay.

FIG. 12 shows the internalization-mediated cellular toxicities of chimeric antibody-DT3C conjugates on 293F-TROP2 cells.

FIG. 13 shows the binding capability of huA1E4F7D4-V16 to human TROP2 in a capture ELISA.

FIG. 14 shows the binding capability of huA1E4F7D4-V16 to cynomolgus TROP2 in an indirect ELISA.

FIG. 15 shows the binding capability of huA1E4F7D4-V16 to 293F-TROP2 cells expressing human TROP2 in a cell based binding FACS assay.

FIG. 16 shows the ability of antibody huA1E4F7D4-V16 to block benchmark-human TROP2 binding in a competitive ELISA test.

FIG. 17 shows the internalization-mediated cellular toxicity of huA1E4F7D4-V16-DT3C conjugate on 293F-TROP2 cells.

FIG. 18 shows the protein thermal shift assay result of huA1E4F7D4-V16.

FIG. 19 shows the binding capability of huA1E4F7D4-V16 to 293F-TROP2 cells expressing human TROP2 in a cell based binding FACS assay.

FIG. 20 shows the internalization-mediated cellular toxicity of the huA1E4F7D4-V16-DT3C conjugate on 293F-TROP2 cells.

### DETAILED DESCRIPTION OF THE INVENTION

To ensure that the present disclosure may be more readily understood, certain terms are first defined. Additional definitions are set forth throughout the detailed description.

The term “TROP2” refers to tumor-associated calcium signal transducer 2, also known as epithelial glycoprotein-1, gastrointestinal antigen 733-1 and membrane component surface marker-1. The term “TROP2” may comprise variants, isoforms, homologs, orthologs and paralogs. For example, an antibody specific for a human TROP2 protein may, in certain cases, cross-react with a TROP2 protein from a species other than human, such as monkey. In other embodiments, an antibody specific for a human TROP2 protein may be completely specific for the human TROP2 protein and exhibit no cross-reactivity to other species or of other types, or may cross-react with TROP2 from certain other species but not all other species.

The term “human TROP2” refers to a TROP2 protein having an amino acid sequence from a human, such as the amino acid sequence of human TROP2 set forth in SEQ ID NO: 71. The terms “monkey TROP2” or “cynomolgus TROP2” refer to a TROP2 protein having an amino acid sequence from macaca nemestrina or macaca mulatta, such as the amino acid sequence having NCBI Accession No. XP_001114599.1 or XP_011762693.1.

The term “antibody” as used herein in some instances refers to an immunoglobulin molecule that recognizes and specifically binds a target, through at least one antigen-binding site wherein the antigen-binding site is usually within the variable region of the immunoglobulin molecule. As used herein, the term encompasses intact polyclonal antibodies, intact monoclonal antibodies, single-chain Fv (scFv) antibodies, heavy chain antibodies (HCAbs), light chain antibodies (LCAbs), multispecific antibodies, bispecific antibodies, monospecific antibodies, monovalent antibodies, fusion proteins comprising an antigen-binding site of an antibody, and any other modified immunoglobulin molecules comprising an antigen-binding site (e.g., dual variable domain immunoglobulin molecules) as long as the antibodies exhibit the desired biological activity. Antibodies also include, but are not limited to, mouse antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes. Unless expressly indicated otherwise, the term “antibody” as used herein include “antigen-binding portion” of the intact antibodies. An IgG is a glycoprotein which may comprise two heavy (H) chains and two light (L) chains inter-connected by disulfide bonds. Each heavy chain may be comprised of a heavy chain variable region (abbreviated herein as V_{H}) and a heavy chain constant region. The heavy chain constant region may be comprised of three domains. C_{H1}, C_{H2} and C_{H3}. Each light chain may be comprised of a light chain variable region (abbreviated herein as V_{L}) and a light chain constant region. The light chain constant region may be comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A “functional fragment” of a heavy chain constant region refers to a part of the constant region that retains the whole-length constant region's functions such as the ability of mediating the binding of the antibody to immune cells and/or complement system proteins. A “functional fragment” of a light chain constant region refers to a part of the constant region that retains the whole-length constant region's functions.

The term “antigen-binding portion” or “antigen-binding fragment” as used in connection with an antibody refers to one or more fragments of an antibody that retain the ability to specifically bind to an antigen (e.g., SARS-CoV-2 spike protein). It has been shown that the antigen-binding function of an antibody can be performed by fragments of a full-length antibody. Examples of binding fragments encompassed within the term “antigen-binding portion” of an antibody include, but not limited to, (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR); and (viii) a nanobody, a heavy chain variable region containing a single variable domain and two constant domains. Furthermore, although the two domains of the Fv fragment, V_{L} and V_{H}, are coded by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., (1988) Science 242:423-426; and Huston et al., (1988) Proc. Natd. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated” antibody or antigen-binding portion thereof, as used herein, is intended to refer to an antibody or an antigen-binding portion thereof that is substantially free of other antibodies having different antigenic specificities (e.g., an isolated antibody that specifically binds a TROP2 protein is substantially free of antibodies that specifically bind antigens other than TROP2 proteins). An isolated antibody or an antigen-binding portion thereof that specifically binds a human TROP2 protein may, however, have cross-reactivity to other antigens, such as TROP2 proteins from other species. Moreover, an isolated antibody can be substantially free of other cellular material and/or chemicals.

The term “mouse antibody”, as used herein, is intended to include antibodies having variable regions in which both the framework and CDR regions are derived from mouse germline immunoglobulin sequences. Furthermore, if the antibody contains a constant region, the constant region also is derived from mouse germline immunoglobulin sequences. The mouse antibodies of the disclosure can include amino acid residues not encoded by mouse germline immunoglobulin sequences (e.g., mutations introduced by random or site-specific mutagenesis in vitro or by somatic mutation in vivo). However, the term “mouse antibody”, as used herein, is not intended to include antibodies in which CDR sequences derived from the germline of another mammalian species have been grafted onto mouse framework sequences.

The term “chimeric antibody” refers to an antibody made by combining genetic material from a nonhuman source with genetic material from a human being. Or more generally, a chimeric antibody is an antibody having genetic material from a certain species with genetic material from another species.

The term “humanized antibody”, as used herein, refers to an antibody from non-human species whose protein sequences have been modified to increase similarity to antibody variants produced naturally in humans.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method.

The term “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

As used herein, an antibody that “specifically binds to human TROP2” is intended to refer to an antibody that binds to human TROP2 protein (and possibly a TROP2 protein from one or more non-human species) but does not substantially bind to non-TROP2 proteins. Preferably, the antibody binds to human TROP2 protein with “high affinity”, namely with a K_{D} of 5.0×10⁻⁸ M or less, more preferably 1.0×10⁻⁸ M or less, and more preferably 2.0×10⁻⁹ M or less.

The term “does not substantially bind” to a protein or cells, as used herein, means does not bind or does not bind with a high affinity to the protein or cells, i.e., binds to the protein or cells with a K_{D} of 1.0×10⁻⁶ M or more, more preferably 1.0×10⁻⁵ M or more, more preferably 1.0×10⁻⁴ M or more, more preferably 1.0×10⁻³ M or more, even more preferably 1.0×10⁻² M or more.

The term “high affinity” for an IgG antibody refers to an antibody having a K_{D} of 1.0×10⁻⁶ M or less, more preferably 5.0×10⁻⁸ M or less, even more preferably 1.0×10⁻⁸ M or less, even more preferably 1.0×10⁻⁹ M or less and even more preferably 5.0×10⁻¹⁰ M or less for a target antigen. However. “high affinity” binding can vary for other antibody isotypes. For example, “high affinity” binding for an IgM isotype refers to an antibody having a K_{D} of 10⁻⁶ M or less, more preferably 10⁻⁷ M or less, even more preferably 10⁻⁸ M or less.

The term “K_{assoc}” or “Kₐ”, as used herein, is intended to refer to the association rate of a particular antibody-antigen interaction, whereas the term “K_{dis}” or “K_{d}”, as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The term “EC₅₀”, also known as half maximal effective concentration, refers to the concentration of an antibody or an antigen-binding portion thereof which induces a response halfway between the baseline and the maximum after a specified exposure time.

The term “IC₅₀”, also known as half maximal inhibitory concentration, refers to the concentration of an antibody or an antigen-binding portion thereof which inhibits a specific biological or biochemical function by 50% relative to the absence of the antibody or antigen-binding portion thereof.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “therapeutically effective amount” means an amount of the antibody or the antigen binding portion of the present disclosure sufficient to prevent or ameliorate the symptoms associated with a disease or condition (such as a tumor) and/or lessen the severity of the disease or condition. A therapeutically effective amount is understood to be in context to the condition being treated, where the actual effective amount is readily discerned by those of skill in the art.

The antibody, or the antigen-binding portion thereof, of the disclosure specifically binds to human TROP2 with comparable, if not higher, binding affinity/capability to human and/or monkey TROP2, and has higher or lower internalization activity, as compared to prior art anti-TROP2 antibodies such as sacituzumab (the antibody part of IMMU-132).

The antibodies or antigen-binding portions thereof of the disclosure are mouse, chimeric and humanized.

The antibody or antigen-binding portion thereof of the disclosure is the monoclonal antibody structurally and chemically characterized as described below and in the following Examples. The amino acid sequence ID numbers of the heavy/light chain variable regions and CDRs of the disclosure are summarized in Table 1 below, some antibodies sharing the same V_{H} or V₁. The heavy chain constant region for the antibodies may be human IgG1 heavy chain constant region having the amino acid sequence set forth in, e.g., SEQ ID NO: 64 (X1=R, X2=E, X3=M; X1=K, X2=D, X3=L), or a functional fragment thereof, and the light chain constant region for the antibodies may be human kappa constant region having an amino acid sequence set forth in, e.g., SEQ ID NO: 65. The antibodies of the disclosure may also contain human IgG2 or IgG4 heavy chain constant region. The antibodies of the disclosure may also contain human kappa light chain constant region.

The heavy chain variable region CDRs and the light chain variable region CDRs in Table 1 have been defined by the Kabat numbering system. However, as is well known in the art, CDR regions can also be determined by other systems such as Chothia, and IMGT, AbM, or Contact numbering system/method, based on heavy chain/light chain variable region sequences.

TABLE 1
Amino acid sequence ID numbers of heavy/light chain variable regions and CDRs of Ig antibodies

| | VH- | VH- | VH- | | VL- | VL- | VL- | |
|---|---|---|---|---|---|---|---|---|
| Antibody ID | CDR1 | CDR2 | CDR3 | VH | CDR1 | CDR2 | CDR3 | VL |
| A1E4F7D4 | 1 | 2 | 3 | 44 | 1 | 5 | 6 | 48 |
| A1E4F7D4-V1 | 1 | 2 | 3 | 45 | 4 | 5 | 6 | 49, X1 = D, X2 = L, X3 = V |
| A1E4F7D4-V2 | 1 | 2 | 3 | 46, X1 = S, X2 = A | 4 | 5 | 6 | 49, X1 = E, X2 = V, X3 = L |
| A1E4F7D4-V3 | 1 | 2 | 3 | 46, X1 = T, X2 = A | 4 | 5 | 6 | 49, X1 = E, X2 = V, X3 = L |
| A1E4F7D4-V4 | 1 | 2 | 3 | 46, X1 = S, X2 = V | 4 | 5 | 6 | 49, X1 = E, X2 = V, X3 = L |
| A1E4F7D4-V5 | 1 | 2 | 3 | 47, X1 = R, X2 = R | 4 | 5 | 6 | 49, X1 = E, X2 = V, X3 = L |
| A1E4F7D4-V6 | 1 | 2 | 3 | 47, X1 = A, X2 = T | 4 | 5 | 6 | 49, X1 = E, X2 = V, X3 = L |
| A1E4F7D4-V7 | 1 | 2 | 3 | 46, X1 = S, X2 = A | 4 | 5 | 6 | 50, X1 = Q, X2 = S, X3 = K |
| A1E4F7D4-V8 | 1 | 2 | 3 | 46, X1 = T, X2 = A | 4 | 5 | 6 | 50, X1 = Q, X2 = S, X3 = K |
| A1E4F7D4-V9 | 1 | 2 | 3 | 46, X1 = S, X2 = V | 4 | 5 | 6 | 50, X1 = Q, X2 = S, X3 = K |
| A1E4F7D4-V10 | 1 | 2 | 3 | 47, X1 = R, X2 = R | 4 | 5 | 6 | 50, X1 = Q, X2 = S, X3 = K |
| A1E4F7D4-V11 | 1 | 2 | 3 | 47, X1 = A, X2 = T | 4 | 5 | 6 | 50, X1 = Q, X2 = S, X3 = K |
| A1E4F7D4-V12 | 1 | 2 | 3 | 46, X1 = S, X2 = A | 4 | 5 | 6 | 50, X1 = G, X2 = A, X3 = K |
| A1E4F7D4-V13 | 1 | 2 | 3 | 46, X1 = T, X2 = A | 4 | 5 | 6 | 50, X1 = G, X2 = A, X3 = K |
| A1E4F7D4-V14 | 1 | 2 | 3 | 46, X1 = S, X2 = V | 4 | 5 | 6 | 50, X1 = G, X2 = A, X3 = K |
| A1E4F7D4-V15 | 1 | 2 | 3 | 47, X1 = R, X2 = R | 4 | 5 | 6 | 50, X1 = G, X2 = A, X3 = K |
| A1E4F7D4-V16 | 1 | 2 | 3 | 47, X1 = A, X2 = T | 4 | 5 | 6 | 50, X1 = G, X2 = A, X3 = K |
| A1E4F7D4-V17 | 1 | 2 | 3 | 46, X1 = S, X2 = A | 4 | 5 | 6 | 50, X1 = G, X2 = S, X3 = Y |
| A1E4F7D4-V18 | 1 | 2 | 3 | 46, X1 = T, X2 = A | 4 | 5 | 6 | 50, X1 = G, X2 = S, X3 = Y |
| A1E4F7D4-V19 | 1 | 2 | 3 | 46, X1 = S, X2 = V | 4 | 5 | 6 | 50, X1 = G, X2 = S, X3 = Y |
| A1E4F7D4-V20 | 1 | 2 | 3 | 47, X1 = R, X2 = R | 4 | 5 | 6 | 50, X1 = G, X2 = S, X3 = Y |
| A1E4F7D4-V21 | 1 | 2 | 3 | 47, X1 = A, X2 = T | 4 | 5 | 6 | 50, X1 = G, X2 = S, X3 = Y |
| A1E11A12D1 | 7 | 8 | 3 | 51 | 9 | 10 | 11 | 52 |
| A1H3C5H8E12 | 12 | 13 | 14 | 53 | 15 | 16 | 17 | 54 |
| A1F1G12A7 | 18 | 19 | 20 | 55 | 21 | 22 | 23 | 56 |
| A1B12D2B4E7B3 | 24 | 25 | 26 | 57 | 27 | 28 | 29 | 58 |
| B1G1F5A3 | 30 | 31 | 32 | 59 | 33 | 34 | 29 | 60 |
| C1B3B12D2 | 35 | 36 | 37 | 61 | 38 | 39 | 40 | 62 |

The amino acid sequences of SEQ ID NOs: 5, 10 and 16 are the same.

The V_{H} and/or V_{L} sequences (or CDR sequences) of other Anti-TROP2 antibodies which bind to human TROP2 can be “mixed and matched” with the V_{H} and/or V_{L} sequences (or CDR sequences) of the anti-TROP2 antibody of the present disclosure. Preferably, in some embodiments with immunoglobulin-like antibodies, when V_{H} and V_{L} chains (or the CDRs within such chains) are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one embodiment, an antibody of the disclosure, or an antigen binding portion thereof, may comprise:
(a) a heavy chain variable region which may comprise an amino acid sequence listed above in Table 1; and/or
(b) a light chain variable region which may comprise an amino acid sequence listed above in Table 1, or the V_{L} of another anti-TROP2 antibody, wherein the antibody specifically binds human TROP2.

In another embodiment, an antibody of the disclosure, or an antigen binding portion thereof, may comprise:
(a) the CDR1, CDR2, and CDR3 regions of the heavy chain variable region listed above in Table 1; and/or
(b) the CDR1, CDR2, and CDR3 regions of the light chain variable region listed above in Table 1 or the CDRs of another anti-TROP2 antibody, wherein the antibody specifically binds human TROP2.

In yet another embodiment, the antibody, or antigen binding portion thereof, includes the heavy chain variable CDR2 region of anti-TROP2 antibody combined with CDRs of other antibodies which bind human TROP2, e.g., CDR1 and/or CDR3 from the heavy chain variable region, and/or CDR1, CDR2, and/or CDR3 from the light chain variable region of a different anti-TROP2 antibody.

In addition, it is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence.

Accordingly, in another embodiment, antibodies of the disclosure may comprise the CDR2 of the heavy chain variable region of the anti-TROP2 antibody and at least the CDR3 of the heavy and/or light chain variable region of the anti-TROP2 antibody, or the CDR3 of the heavy and/or light chain variable region of another anti-TROP2 antibody, wherein the antibody is capable of specifically binding to human TROP2. These antibodies preferably (a) compete for binding with TROP2; (b) retain the functional characteristics; (c) bind to the same epitope; and/or (d) have a similar binding affinity as the anti-TROP2 antibody of the present disclosure. In yet another embodiment, the antibodies further may comprise the CDR2 of the light chain variable region of the anti-TROP2 antibody, or the CDR2 of the light chain variable region of another anti-TROP2 antibody, wherein the antibody is capable of specifically binding to human TROP2. In another embodiment, the antibodies of the disclosure may include the CDR1 of the heavy and/or light chain variable region of the anti-TROP2 antibody, or the CDR1 of the heavy and/or light chain variable region of another anti-TROP2 antibody, wherein the antibody is capable of specifically binding to human TROP2.

In another embodiment, an antibody or an antigen-binding portion thereof of the disclosure may comprise a heavy and/or light chain variable region sequences of CDR1, CDR2 and CDR3 sequences which differ from those of the anti-TROP2 antibodies of the present disclosure by one or more conservative modifications. It is understood in the art that certain conservative sequence modification can be made which do not remove antigen binding.

Accordingly, in one embodiment, the antibody may comprise a heavy chain variable region which may comprise CDR1, CDR2, and CDR3 sequences and/or a light chain variable region which may comprise CDR1, CDR2, and CDR3 sequences, wherein:
(a) the heavy chain variable region CDR1 sequence may comprise a sequence listed in Table 1 above, and/or conservative modifications thereof; and/or
(b) the heavy chain variable region CDR2 sequence may comprise a sequence listed in Table 1 above, and/or conservative modifications thereof; and/or
(c) the heavy chain variable region CDR3 sequence may comprise a sequence listed in Table 1 above, and conservative modifications thereof; and/or
(d) the light chain variable region CDR1, and/or CDR2, and/or CDR3 sequences may comprise the sequence(s) listed in Table 1 above; and/or conservative modifications thereof; and
(e) the antibody specifically binds human TROP2.

In various embodiments, the antibody or antigen-binding portion thereof can be, for example, mouse, chimeric, or humanized.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the disclosure can be replaced with other amino acid residues from the same side chain family and the altered antibody can be tested for retained function (i.e., the functions set forth above) using the functional assays described herein.

Antibodies of the disclosure can be prepared using an antibody having one or more of the V_{H}/V_{L} sequences of the anti-TROP2 antibody of the present disclosure as starting material to engineer a modified antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad. See also U.S.A. 86:10029-10033; U.S. Pat. Nos. 5,225,539; 5,530,101; 5,585,089; 5,693,762 and 6,180,370).

Framework sequences can be obtained from public DNA databases or published references that include germline antibody gene sequences.

Antibody protein sequences are compared against a compiled protein sequence database using one of the sequence similarity searching methods called the Gapped BLAST (Altschul et al., (1997), supra), which is well known to those skilled in the art. Preferred framework sequences for use in the antibodies of the disclosure are those that are structurally similar to the framework sequences used by antibodies of the disclosure.

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as known in the art. Preferably conservative modifications (as known in the art) are introduced. The mutations can be amino acid substitutions, additions or deletions, but are preferably substitutions. Moreover, typically no more than one, two, three, four or five residues within a CDR region are altered.

Engineered antibodies of the disclosure include those in which modifications have been made to framework residues within V_{H} and/or V_{L}, e.g., to improve the properties of the antibody. Typically, such framework modifications are made to decrease the immunogenicity of the antibody. For example, one approach is to “back-mutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043.

In addition, or as an alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the disclosure can be chemically modified (e.g., one or more chemical moieties can be attached to the antibody) or be modified to alter its glycosylation, again to alter one or more functional properties of the antibody.

In another embodiment, the Fc hinge region of an antibody is mutated to decrease the biological half-life of the antibody. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of an antibody is modified. For example, a glycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. See, e.g., U.S. Pat. Nos. 5,714,350 and 6,350,861.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase or reduce the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation.

Another modification of the antibodies herein that is contemplated by this disclosure is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the disclosure. See, e.g., EP 0 154 316 and EP 0 401 384.

Antibodies of the disclosure can be characterized by their various physical properties, to detect and/or differentiate different classes thereof.

For example, antibodies can contain one or more glycosylation sites in either the light or heavy chain variable region. Such glycosylation sites may result in increased immunogenicity of the antibody or an alteration of the pK of the antibody due to altered antigen binding (Marshall et al (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al., (2000) Mol Immunol 37:697-706). Glycosylation has been known to occur at motifs containing an N—X—S/T sequence.

In a preferred embodiment, the antibodies do not contain asparagine isomerism sites. The deamidation of asparagine may occur on N-G or D-G sequences and result in the creation of an isoaspartic acid residue that introduces a link into the polypeptide chain and decreases its stability (isoaspartic acid effect).

Each antibody will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8. There is speculation that antibodies with a pI outside the normal range may have some unfolding and instability under in vivo conditions. Thus, it is preferred to have an anti-TROP2 antibody that contains a pI value that falls in the normal range. This can be achieved either by selecting antibodies with a pI in the normal range or by mutating charged surface residues.

In another aspect, the disclosure provides nucleic acid molecules that encode heavy and/or light chain variable regions, or CDRs, of the antibodies of the disclosure. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques. A nucleic acid of the disclosure can be, e.g., DNA or RNA and may or may not contain intronic sequences. In a preferred embodiment, the nucleic acid is a cDNA molecule.

Nucleic acids of the disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

Preferred nucleic acids molecules of the disclosure include those encoding the V_{H} and/or V_{L} sequences of the TROP2 monoclonal antibody or the CDRs. Once DNA fragments encoding V_{H} and/or V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (C_{H1}, C_{H2} and C_{H3}). The sequences of human heavy chain constant region genes are known in the art and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain C_{H1} constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker (see e.g., Bird et al., (1988) Science 242:423-426; Huston et al., (1988) Proc. Natl. Acad. Sci. USA 85:5879-5883; McCafferty et al., (1990) Nature 348:552-554).

Monoclonal antibodies (mAbs) of the present disclosure can be produced using the well-known somatic cell hybridization (hybridoma) technique of Kohler and Milstein (1975) Nature 256: 495. Other embodiments for producing monoclonal antibodies include viral or oncogenic transformation of B lymphocytes and phage display techniques. Chimeric or humanized antibodies are also well known in the art.

Antibodies of the disclosure also can be produced in a host cell transfectoma using, for example, a combination of recombinant DNA techniques and gene transfection methods as is well known in the art (e.g., Morrison, S. (1985) Science 229:1202). In one embodiment, DNA encoding partial or full-length light and heavy chains obtained by standard molecular biology techniques is inserted into one or more expression vectors such that the genes are operatively linked to transcriptional and translational regulatory sequences. In this context, the term “operatively linked” is intended to mean that an antibody gene is ligated into a vector such that transcriptional and translational control sequences within the vector serve their intended function of regulating the transcription and translation of the antibody gene.

The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyomavirus enhancer. Alternatively, non-viral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al.. (1988) Mol. Cell. Biol. 8:466-472). The expression vector and expression control sequences are chosen to be compatible with the expression host cell used.

The antibody light chain gene and the antibody heavy chain gene can be inserted into the same or separate expression vectors. In preferred embodiments, the variable regions are used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes and regulatory sequences, the recombinant expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the heavy and/or light chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Preferred mammalian host cells for expressing the recombinant antibodies of the disclosure include Chinese Hamster Ovary (CHO cells) (including dhfr-CHO cells, described in Urlaub and Chasin, (1980) Proc. Natd. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

In another aspect, the present disclosure features bispecific molecules which may comprise one or more antibodies of the disclosure linked to at least one other functional molecule, e.g., another peptide or protein (e.g., another antibody or ligand for a receptor) to generate a bispecific molecule that binds to at least two different binding sites or target molecules. Thus, as used herein. “bispecific molecule” includes molecules that have three or more specificities. In an embodiment, a bispecific molecule has, in addition to the FcR binding specificity and an anti-TROP2 binding specificity, a third specificity. The bispecific molecule of the disclosure may be in certain embodiments engineered to have reduced FcR binding affinity.

Bispecific molecules may be in many different formats and sizes. At one end of the size spectrum, a bispecific molecule retains the traditional antibody format, except that, instead of having two binding arms of identical specificity, it has two binding arms each having a different specificity. At the other extreme are bispecific molecules consisting of two single-chain antibody fragments (scFv's) linked by a peptide chain, a so-called Bs(scFv) 2 construct. Intermediate-sized bispecific molecules include two different F(ab) fragments linked by a peptidyl linker. Bispecific molecules of these and other formats can be prepared by genetic engineering, somatic hybridization, or chemical methods.

Antibodies or antigen-binding portions thereof of the disclosure can be conjugated to a therapeutic agent to form an immunoconjugate such as an antibody-drug conjugate (ADC). Suitable therapeutic agents include an anti-inflammatory agent and an anti-cancer agent. In the ADC, the antibody and therapeutic agent preferably are conjugated via a linker cleavable such as a peptidyl, disulfide, or hydrazone linker. More preferably, the linker is a peptidyl linker such as Val-Cit. Ala-Val. Val-Ala-Val, Lys-Lys, Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038,658: WO 07/051,081; WO 07/059,404; WO 08/083,312; and WO 08/103,693; U.S. Patent Publications 20060024317; 20060004081; and 20060247295; the disclosures of which are incorporated herein by reference.

An oncolytic virus preferentially infects and kills cancer cells. Antibodies of the present disclosure can be used in conjunction with oncolytic viruses. Alternatively, oncolytic viruses encoding antibodies of the present disclosure can be introduced into human body.

Also provided herein are a chimeric antigen receptor (CAR) containing an anti-TROP2 scFv or V_{H}H fragment, the anti-TROP2 scFv or V_{H}H may comprise CDRs and heavy/light chain variable regions described herein.

The anti-TROP2 CAR may comprise (a) an extracellular antigen binding domain which may comprise an anti-TROP2 scFv or V_{H}H; (b) a transmembrane domain; and (c) an intracellular signaling domain. The CAR may contain a signal peptide at the N-terminus of the extracellular antigen binding domain that directs the nascent receptor into the endoplasmic reticulum, and a hinge peptide at the N-terminus of the extracellular antigen binding domain that makes the receptor more available for binding. The CAR preferably comprises, at the intracellular signaling domain, a primary intracellular signaling domain and one or more co-stimulatory signaling domains. The mainly used and most effective primary intracellular signaling domain is CD3-zeta cytoplasmic domain which contains ITAMs, the phosphorylation of which results in T cell activation. The co-stimulatory signaling domain may be derived from the co-stimulatory proteins such as CD28, CD137 and OX40. The CARs may further add factors that enhance T cell expansion, persistence, and anti-tumor activity, such as cytokines, and co-stimulatory ligands.

Also provided are engineered immune effector cells, which may comprise the CAR provided herein. In certain embodiments, the immune effector cell is a T cell, an NK cell, a peripheral blood mononuclear cell (PBMC), a hematopoietic stem cell, a pluripotent stem cell, or an embryonic stem cell. In certain embodiments, the immune effector cell is a T cell.

In another aspect, the present disclosure provides a pharmaceutical composition which may comprise the antibody or antigen-binding portion thereof, the bispecific molecule, the CAR-T cell, the oncolytic virus, the immunoconjugate, or alternatively the nucleic acid molecule, the expression vector or the host cell, of the disclosure, formulated together with a pharmaceutically acceptable carrier. The antibody or antigen-binding portion thereof, the bispecific molecule, the CAR-T cell, the oncolytic virus, the immunoconjugate, the nucleic acid molecule, the expression vector or the host cell can be dosed separately when the composition contains more than one kind of molecules. The composition may optionally contain one or more additional pharmaceutically active ingredients, such as an anti-tumor drug.

The pharmaceutical composition may comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy. 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Alternatively, an antibody of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a micro-emulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

For administration of the composition, the dosage may range from about 0.0001 to 100 mg/kg. An exemplary treatment regime entails administration once a month.

A “therapeutically effective dosage” of an anti-TROP2 antibody, or the antigen-binding portion thereof, the bispecific molecule, the CAR-T cell, the oncolytic virus, the immunoconjugate, the nucleic acid molecule, the expression vector, or the host cell, of the disclosure preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably eliminate inflammations by at least about 20%, more preferably by at least about 40%, even more preferably by at least about 60%, and still more preferably by at least about 80% relative to untreated subjects.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the monoclonal antibodies of the disclosure can be formulated to ensure proper distribution in vivo. For example, to ensure that the therapeutic antibody or antigen-binding portion thereof of the disclosure cross the blood-brain barrier, they can be formulated in liposomes, which may additionally comprise targeting moieties to enhance selective transport to specific cells or organs. See, e.g. U.S. Pat. Nos. 4,522,811; 5,374,548; 5,416,016; and 5,399,331; V. V. Ranade (1989) J. Clin. Pharmacol. 29:685; Umezawa et al., (1988) Biochem. Biophys. Res. Commun. 153:1038; Bloeman et al., (1995) FEBS Lett. 357:140; M. Owais et al., (1995) Antimicrob. Agents Chemother. 39:180; Briscoe et al., (1995) Am. J. Physiol. 1233:134; Schreier et al., (1994) J. Biol. Chem. 269:9090; Keinanen and Laukkanen (1994) FEBS Lett. 346:123; and Killion and Fidler (1994) Immunomethods 4:273.

The pharmaceutical composition of the present disclosure have numerous in vitro and in vivo utilities involving, for example, treatment of tumors with excessive TROP2 signaling.

Given that the TROP2 is associated with tumor cell proliferation, the disclosure provides methods for treating TROP2 related tumors or cancers in a subject in need thereof, which may comprise administering to the subject the pharmaceutical composition of the disclosure. The tumor may be a solid tumor or a hematological tumor, including, but not limited to, breast cancer, colorectal cancer, gastric adenocarcinoma, esophageal cancer, hepatocellular carcinoma, non-small-cell lung cancer, small-cell lung cancer, ovarian epithelial cancer, prostate cancer, pancreatic ductal adenocarcinoma, head and neck cancer, squamous cell cancer, renal cell cancer, urinary bladder neoplasm, cervical cancer, endometrial cancer, follicular thyroid cancer, and glioblastoma multiforme. In certain embodiments, at least one additional anti-cancer antibody may be further administered. In certain embodiments, the subject is human.

In another aspect, the disclosure provides methods of combination therapy in which the pharmaceutical composition of the present disclosure is co-administered with one or more additional antibodies that are effective in inhibiting tumor growth in a subject. In one embodiment, the disclosure provides a method for inhibiting tumor growth in a subject which may comprise administering to the subject the pharmaceutical composition of the disclosure and one or more additional antibodies, such as an anti-OX40 antibody, an anti-TIM-3 antibody, an anti-CD137 antibody, an anti-GITR antibody, an anti-LAG-3 antibody, an anti-PD-L1 antibody, and anti-PD-1 antibody. In certain embodiments, the subject is human. The TROP2 pathway blockade can also be further combined with standard cancer treatments.

In yet another aspect, the disclosure provides diagnostic methods, compositions and kits. In an embodiment, an antibody or an antigen-binding portion of the disclosure is used to determine the presence and expression of TROP2 in a tissue. In an embodiment, the diagnostic indicates prognosis and/or directs treatment and/or follow-up treatment. For example, TROP2 signaling can be targeted for treatment of tumors. In an embodiment, an antibody or an antigen binding portion of the disclosure is employed in diagnostic kit or method to determine prognosis and appropriate treatment and follow-up of TROP2 related tumors or cancers.

The combination of therapeutic agents discussed herein can be administered concurrently as a single composition in a pharmaceutically acceptable carrier, or concurrently as separate compositions with each agent in a pharmaceutically acceptable carrier. In another embodiment, the combination of therapeutic agents can be administered sequentially.

Furthermore, if more than one dose of the combination therapy is administered sequentially, the order of the sequential administration can be reversed or kept in the same order at each time point of administration, sequential administrations can be combined with concurrent administrations, or any combination thereof.

The disclosure further provides a method for imaging of TROP2-positive tissues, e.g., cancer tissues, in a subject in need thereof, comprising administering the subject with a radioactively labeled anti-TROP2 antibody or antigen-binding portion thereof, the immunoconjugate, or the bispecific molecule of the disclosure. The method may be used to trace/detect the distribution of a tumor or cancer with high TROP2 expression, including, but not limited to, esophageal squamous cell carcinoma, colorectal cancer, pancreatic cancer, colon cancer, papillary thyroid cancer, breast cancer, and bladder cancer. In certain embodiments, the subject is human.

Although the present invention and its advantages have been described in detail, it should be understood that various changes, substitutions and alterations can be made herein without departing from the spirit and scope of the invention as defined in the appended claims.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1 Generation of Mouse Anti-TROP2 Monoclonal Antibodies

### Immunization

Mice were immunized according to the method as described in E Harlow, D. Lane, Antibody: A Laboratory Manual, Cold Spring Harbor Laboratory Press, Cold Spring Harbor, N.Y., 1998. In house made recombinant human TROP2 protein with human IgG1 Fc at the C-terminus (amino acid sequence set forth in SEQ ID NO: 66) was used as the immunogen, and in house made human TROP2-his protein (amino acid sequence set forth in SEQ ID NO: 67) was used for determining anti-sera titers and for screening hybridomas secreting antigen-specific antibodies.

Immunizing dosages contained 20 μg human TROP2-Fc proteins per mouse per injection for both the primary and boost immunizations. To increase immune responses, the complete Freud's adjuvant and incomplete Freud's adjuvant (Sigma, St. Louis, Mo., USA) were used respectively for primary and boost immunizations. Briefly, adjuvant-antigen mixture was prepared as follows. First, the adjuvant was gently mixed in a vial using a vortex, and the desired amount of adjuvant was transferred to an autoclaved 1.5 mL micro-centrifuge tube. The antigen was prepared in PBS or saline with the concentration ranging from 0.2-0.27 mg/ml, and the calculated amount of antigen was then added to the micro-centrifuge tube with the adjuvant. The resulting mixtures were mixed by gently vortexing for 2 minutes to generate water-in-oil emulsions. The adjuvant-antigen emulsions were then drawn into the proper syringe for animal injection. A total of 20 μg of antigen was injected in a volume of 150-200 μl. Each animal was immunized, and then boosted for 4 to 5 times depending on the anti-sera titers. Animals with good titers were given a final boost by intraperitoneal injection before fusion.

### Hybridoma Fusion and Screening

Cells of murine myeloma cell line (SP2/0-Ag14, ATCC #CRL-1581) were cultured to reach the log phase stage right before fusion. Spleen cells from immunized mice were prepared sterilely and fused with myeloma cells according to the method as described in Kohler G, and Milstein C, “Continuous cultures of fused cells secreting antibody of predefined specificity,” Nature, 256: 495-497 (1975). Fused “hybrid cells” were subsequently dispensed into 96-well plates in DMEM/20% FCS/HAT medium. Surviving hybridoma colonies were observed under the microscope seven to ten days post fusion. After two weeks, the supernatant from each well was subjected to Capture ELISA using human TROP2-his protein (prepared in-house with SEQ ID NO: 67). Positive hybridomas secreting antibodies that bound to human TROP2 proteins were selected and transferred to 24-well plates. These hybridoma clones were further tested for cynomolgus TROP2 binding activity. Hybridoma clones producing antibodies that showed high specific human TROP2 binding and cynomolgus Trop2 binding activity were subcloned by limited dilution to ensure the clonality of the cell line, and then monoclonal antibodies were purified. Briefly, Protein A sepharose columns (from bestchrom (Shanghai) Biosciences, Cat #AA0273) were washed using PBS buffer in 5 to 10 column volumes. Cell supernatants of hybridoma monoclones were passed through the columns, and then the columns were washed using PBS buffer until the absorbance for protein reached the baseline. The columns were eluted with elution buffer (0.1 M Glycine-HCl, pH 2.7), and immediately collected into 1.5 ml tubes with neutralizing buffer (1 M Tris-HCl, pH 9.0). Fractions containing immunoglobulins were pooled and dialyzed in PBS ovemight at 4° C.

### Example 2 Binding Affinity Determination of Mouse Anti-TROP2 Monoclonal Antibodies Using BIACORE Surface Plasmon Resonance

The purified anti-TROP2 mouse monoclonal antibodies (mAbs) generated in Example 1 were characterized for binding affinity and binding kinetics by Biacore T200 system (GE healthcare, Pittsburgh, PA, USA).

Briefly, goat anti-mouse IgG antibodies (GE healthcare, Cat #BR100838, Mouse Antibody Capture Kit) were covalently linked to a CM5 chip (carboxy methyl dextran coated chip from GE healthcare #BR100530) via primary amines, using a standard amine coupling kit (GE healthcare, Pittsburgh, PA, USA) provided by Biacore, or a Protein G chip (GE healthcare, Cat #29-1793-15), wherein the Protein G chip was for affinity determination of the benchmark (in house prepared sacituzumab, also referred to as BM or BM1 herein, amino acid sequences of the heavy and light chains set forth in SEQ ID NOs: 68 and 69, respectively). Un-reacted moieties on the chip (biosensor) surface were blocked with ethanolamine. The anti-TROP2 antibodies generated in Example 1 and the benchmark, at the concentration of 2 μg/ml, were respectively flowed onto the chips at a flow rate of 10 μL/min. Then, serially diluted human TROP2-his proteins (prepared in-house with SEQ ID NO: 67), or cynomolgus TROP2-his proteins (prepared in-house with SEQ ID NO: 70), 2-fold dilution in HBS-EP⁺ buffer (provided by Biacore) starting at 160 nM, were flowed onto the chip at a flow rate of 30 μL/min. The antigen-antibody association kinetics was followed for 2 minutes and the dissociation kinetics was followed for 10 minutes. The association and dissociation curves were fit to a 1:1 Langmuir binding model using BIAcore evaluation software. The K_{D}, Kₐ and K_{d} values were determined and summarized in Table 2 below.

TABLE 2
Binding affinities of mouse anti-TROP2 antibodies

| | Kinetics on Biacore | | | | | |
|---|---|---|---|---|---|---|
| | Human TROP2 | | | cynomolgus TROP2 | | |
| | Kₐ | K_{d} | K_{D} | Kₐ | K_{d} | K_{D} |
| Mouse mAb ID# | (M⁻¹s⁻¹) | (s⁻¹) | (M) | (M⁻¹s⁻¹) | (s⁻¹) | (M) |
| A1E4F7D4 | 3.73E+05 | 8.33E−08 | 2.24E−13 | 4.83E+05 | 4.48E−09 | 9.27E−15 |
| A1B12D2B4E7B3 | 8.40E+04 | 8.92E−05 | 1.06E−09 | 1.11E+05 | 1.24E−04 | 1.12E−09 |
| A1E11A12D1 | 5.25E+05 | 8.09E−05 | 1.54E−10 | 6.78E+05 | 3.82E−05 | 5.63E−11 |
| A1F1G12A7 | 1.11E+05 | 2.36E−04 | 2.13E−09 | 1.52E+05 | 9.44E−04 | 6.23E−09 |
| A1H3C5H8E12 | 5.31E+05 | 9.61E−04 | 1.81E−09 | 6.74E+05 | 7.58E−04 | 1.12E−09 |
| BM1 | 2.55E+05 | 2.22E−04 | 8.68E−10 | 3.54E+05 | 1.97E−04 | 5.57E−10 |
| B1G1F5A3 | 1.340E+5 | 5.580E−5 | 4.164E−10 | 1.561E+5 | 8.862E−5 | 5.677E−10 |
| BM1 | 2.55E+05 | 2.22E−04 | 8.68E−10 | 3.54E+05 | 1.97E−04 | 5.57E−10 |
| C1B3B12D2 | 4.76E+05 | 6.45E−07 | 1.36E−12 | 5.08E+05 | 9.44E−07 | 1.86E−12 |

All the mouse antibodies of the disclosure specifically bound to human TROP2 and cynomolgus TROP2, at comparable or higher binding affinity as compared to the benchmark. The mouse antibodies A1E4F7D4, A1E11A12D1 and C1B3B12D2 showed the highest binding affinity to human TROP2 and cynomolgus TROP2.

### Example 3 Binding Activity of Mouse Anti-TROP2 Monoclonal Antibodies

The binding activity of mouse anti-TROP2 antibodies of the disclosure to TROP2 was determined by Capture ELISA, Indirect ELISA and Flow Cytometry (FACS).

### Capture ELISA

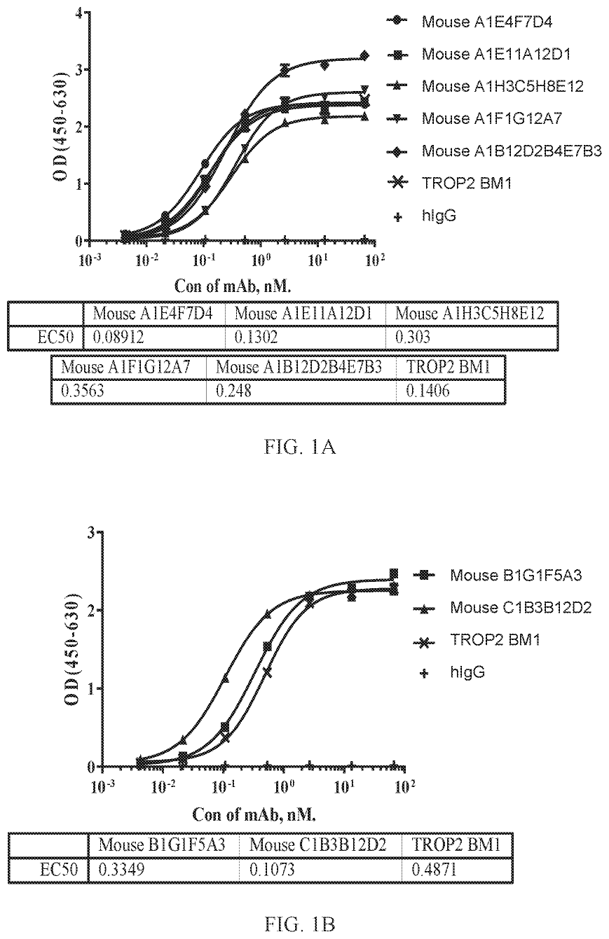

Briefly, 96-well plates were coated with 100 μl 2 μg/ml AffiniPure Goat Anti-Mouse IgG, Fcγ fragment specific (Jackson Immuno Research, Cat #115-005-071) in PBS overnight at 4° C. Plates were washed once with wash buffer (PBS+0.05% v/v Tween-20, PBST) and then blocked with 200 μl/well blocking buffer (5% w/v non-fatty milk in PBST) for 2 hours at 37° C. Plates were washed 4 times and respectively incubated with 100 μl serially diluted anti-TROP2 antibodies of the disclosure, the benchmark or hIgG as a negative control (human immunoglobulin (pH4) for intravenous injection, Hualan Biological Engineering Inc.) (5-fold dilution in PBST containing 2.5% w/v non-fatty milk, starting at 66.7 nM) for 40 minutes at 37° C., and then washed 4 times again. Plates containing captured anti-TROP2 antibodies were incubated with biotin-labeled human TROP2-his protein (prepared in house, SEQ ID NO: 67, 56.7 ng/mL in 2.5% w/v non-fatty milk in PBST, 100 μl/well) for 40 minutes at 37° C., washed 4 times, and incubated with streptavidin conjugated HRP (1:10000 dilution in PBST, Jackson Immuno Research, Cat #016-030-084, 100 μl/well) for 40 minutes at 37° C. After a final wash, plates were incubated with 100 μl/well ELISA substrate TMB (Innoreagents, Cat #TMB-S-002) at room temperature. The reaction was stopped in 3-10 minutes at room temperature with 50 μl/well 1M H₂SO₄, and the absorbance of each well was read on a microplate reader using dual wavelength mode with 450 nm for TMB and 630 nm as the reference wavelength. The OD (450-630) values were plotted against antibody concentration. Data was analyzed using Graphpad Prism software and EC₅₀ values were reported. The results were shown in FIGS. 1A-1B.

### Indirect ELISA

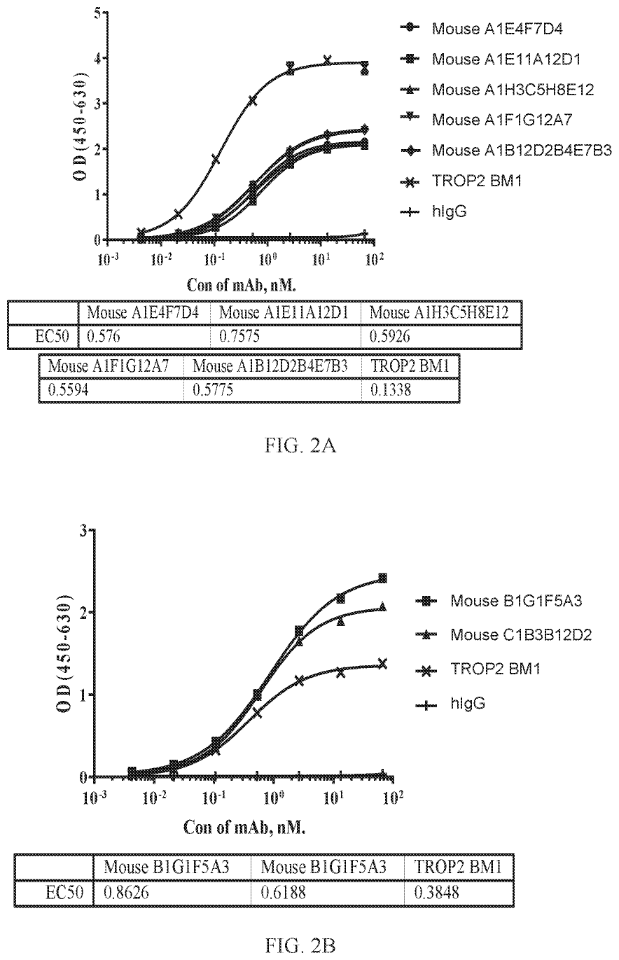

The anti-TROP2 antibodies of the disclosure were tested for their cross-reaction with cynomolgus TROP2 proteins. Briefly, 96-well micro plates were coated with 100 μl 2 μg/ml cynomolgus TROP2-his proteins (prepared in-house with SEQ ID NO: 70) in carbonate/bicarbonate buffer (pH 9.6) overnight at 4° C. ELISA plates were washed once with wash buffer (PBS+0.05% v/v Tween-20, PBST) and then blocked with 200 μl/well blocking buffer (5% w/v non-fatty milk in PBST) for 2 hours at 37° C. Plates were washed 4 times and incubated with 100 μl/well serially diluted anti-TROP2 antibodies of the disclosure or controls (starting at 66.7 nM, 5-fold serial dilution in 2.5% w/v non-fatty milk in PBST) for 40 minutes at 37° C. ELISA plates were washed 4 times again and incubated with Peroxidase AffiniPure Goat Anti-Mouse IgG, Fcγ Fragment Specific (1:5000 dilution in PBST buffer, Jackson Immunoresearch, Cat #115-035-071, 100 μl/well) for 40 minutes at 37° C. After a final wash, plates were incubated with 100 μl/well TMB (Innoreagents) at room temperature. The reaction was stopped 3-10 minutes later at room temperature with 50 μl/well 1M H₂SO₄, and the absorbance of each well was read on a microplate reader using dual wavelength mode with 450 nm for TMB and 630 nm as the reference wavelength. The OD (450-630) values were plotted against antibody concentration. Data was analyzed using Graphpad Prism software and EC₅₀ values were reported. The results were shown in FIGS. 2A-2B.

### Cell-Based Binding FACS

The binding activity of the mouse anti-TROP2 antibodies to cell surface TROP2 proteins was tested by flow cytometry (FACS), using Biosion in-house prepared 293F-TROP2 cells (clone ID #3A8) stably expressing full length human TROP2s (uniprot #P09758, SEQ ID NO.: 71) on cell membrane. The 293F-TROP2 cells were prepared by transfecting 293F cells (Thermofisher Inc., Cat #11625019) with a pCMV-T-P plasmid inserted with human TROP2 coding sequence between EcoRI and XbaI sites, following the instruction of lipofectamine 3000 transfection reagent (Thermo Fisher).

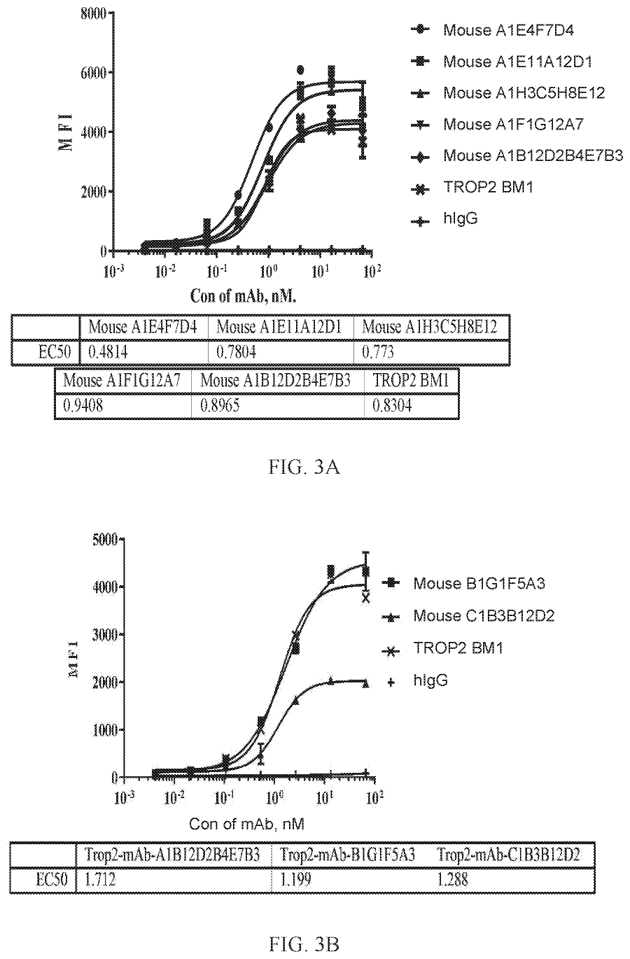

The 293F-TROP2 cells were harvested from cell culture flasks, washed twice and re-suspended in phosphate buffered saline (PBS) containing 2% v/v Fetal Bovine Serum (FACS buffer). Then, 2×10⁵ 293F-TROP2 cells per well were incubated in 96 well-plates with 100 μl of the anti-TROP2 antibodies or controls at various concentrations (starting at 66.7 nM, 4-fold serial dilution in FACS buffer) for 40 minutes on ice. Cells were washed twice with FACS buffer, and added with 100 μL/well R-Phycoerythrin AffiniPure F(ab′)₂ Fragment Goat Anti-Mouse IgG (H+L) (1:1000 dilution in FACS buffer, Jackson ImmunoResearch Laboratories Inc., Cat #115-116-146). Following an incubation of 40 minutes at 4° C. in dark, cells were washed twice and re-suspended in FACS buffer. Fluorescence was measured using a Becton Dickinson FACS Canto II-HTS equipment, and the MFI (mean fluorescence intensity) was plotted against antibody concentration. Data was analyzed using Graphpad Prism software and EC₅₀ values were reported. The results were shown in FIGS. 3A-3B.

It can be seen from FIGS. 1A-1B that all the mouse anti-TROP2 antibodies of the disclosure specifically bound to human TROP2s. The antibodies A1E4F7D4, A1E11A12D1, B1G1F5A3 and C1B3B12D2 showed lower EC₅₀s than that of the benchmark, suggesting that they more efficiently bound to the human TROP2 protein, and the antibody A1B12D2B4E7B3 showed higher Bₘₐₓ than the benchmark. As shown in FIGS. 3A-3B, the mouse anti-TROP2 antibodies A1E4F7D4, A1E11A12D1 and A1H3C5H8E12 showed significantly higher binding capability than the benchmark in the FACS test.

According to FIGS. 2A-2B, all antibodies of the disclosure specifically bound to the monkey TROP2, wherein B1G1F5A3 and C1B3B12D2 bound the monkey TROP2 protein with higher binding activity than the benchmark.

### Example 4 Epitope Binning

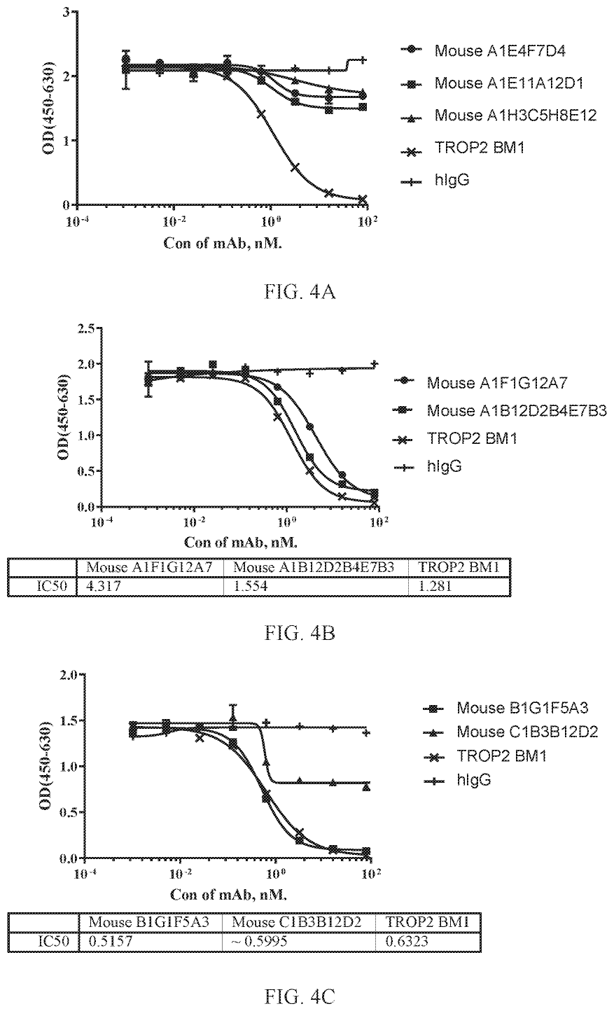

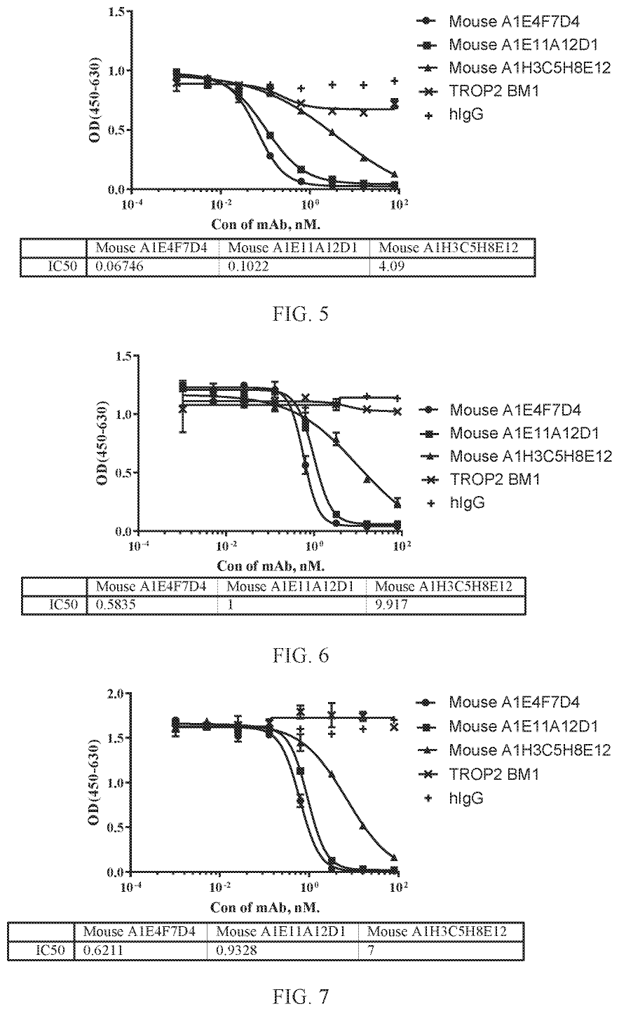

The mouse anti-TROP2 antibodies were tested for epitope binding in a competitive ELISA assay. Briefly, 100 μl of the benchmark at 1 μg/mL, mouse antibody A1E4F7D4 at 2 μg/mL, mouse antibody A1E11A12D1 at 2 μg/mL, and mouse antibody A1H3C5H8E12 at 2 μg/mL, in PBS were respectively coated on 96-well micro plates for 2 hours at 37° C. ELISA plates were washed once with wash buffer (PBS+0.05% v/v Tween-20, PBST) and then blocked with 200 μl blocking buffer (5% w/v non-fatty milk in PBST) for 2 hours at 37° C. While blocking, the anti-TROP2 antibodies or controls were diluted with biotin labeled human TROP2-his protein (SEQ ID NO: 67, 34 ng/mL in 2.5% wA non-fatty milk in PBST), starting at 80 nM with a 5-fold serial dilution, and incubated at room temperature for 40 minutes. After plate washing for 4 times, the antibody/TROP2-his protein mixtures were added to the antibody coated plates, 100 μl per well. After incubation at 37° C. for 40 minutes, plates were washed 4 times again using wash buffer. Then the plates were added and incubated with 100 μl Peroxidase Streptavidin (1:10000 dilution in PBST buffer. Jackson Immunoresearch, Cat #016-030-084) for 40 minutes at 37° C. Plates were washed again using wash buffer. Finally, TMB was added and the reaction was stopped using 1M H₂SO₄. The absorbance of each well was read on a microplate reader using dual wavelength mode with 450 nm for TMB and 630 nm as the reference wavelength, and the OD (450-630) values were plotted against antibody concentration. Data was analyzed using Graphpad Prism software and IC₅₀ values were reported. The capability of the antibodies to block benchmark-TROP2 binding was shown in FIGS. 4A-4C, and the capability of the antibodies to block TROP2 binding with A1E4F7D4, A1E11A12D1 and A1H3C5H8E12 were respectively shown in FIGS. 5-7.

It can be seen from FIGS. 4A-4C that the anti-TROP2 antibodies A1F1G12A7, A1B12D2B4E7B3 and B1G1F5A3 were able to block BM-human TROP2 binding, suggesting that the epitopes they bound and that bound by the benchmark may overlap. The remaining mouse anti-TROP2 antibodies, including A1E4F7D4, A1E11A12D1, A1H3C5H8E12, C1B3B12D2 did not block benchmark binding to human TROP2, suggesting that they might bind to different epitopes as compared to the benchmark.

As shown in FIGS. 5-7, the epitopes bound by A1E4F7D4, A1E11A12D1 and A1H3C5H8E12 overlapped, with the epitopes bound by A1E4F7D4 and A1E11A12D1 spanned more amino acid residues than that by A1H3C5H8E12.

### Example 5 Cell Based Internalization Assay of Anti-TROP2 Antibodies

In the cell-based internalization assay, the anti-TROP2 antibodies were evaluated precisely for their internalization rates using Biosion in-house prepared 293F-TROP2 cells (clone ID #3A8). Firstly a recombinant protein termed DTTP-1170 was synthesized using the amino acid sequence set forth in SEQ ID NO: 72. Then, 5×10′ 293F-TROP2 cells in 100 μL FreeStyle293 medium (Gibco, Cat #12338-018) supplemented with 10% v/v FBS (Gibco, Cat #10099-141) were plated in 96 well-flat bottom plates (Thermo Fisher Scientific Inc., Cat #167008). On the next day of cell seeding, the mouse anti-TROP2 antibodies of the disclosure or controls, 1.6 μg/mL in FreeStyle293 medium with 10% v/v FBS, were mixed with the DTTP1170 proteins, 1.6 μg/mL in FreeStyle293 medium with 10% v/v FBS, at 1:1 volume ratio, and incubated at room temperature for 30 minutes, which were then serially diluted in the cell culture medium, 3-fold serial dilution, starting from 0.8 μg/mL. Then, 100 μl of the serially diluted antibody/DTTP1170 mixtures were added to the cell plates, and incubated in a CO₂ incubator at 37° C. for 72 hours. The plates were added with Cell Titer Glo reagent (Vazyme Biotech Co., Ltd, Cat #DD1101-02) and incubated for 3-5 minutes at room temperature. The cell culture plates were then analyzed by Tecan infinite 200Pro plate-reader. Data were analyzed using Graphpad prism software and IC₅₀ values were reported as the antibody concentrations that achieved 50% of maximal inhibition on cell viability.

When the mAb-DTTP conjugates were internalized by the target cells, target cell viability markedly decreased. If the conjugates were not internalized, then the free DTTP1170 in the medium had no or little cell killing activity. The results were shown in FIG. 8, which showed that DTTP1170 conjugates of all the mouse antibodies of the disclosure, including A1E4F7D4, A1B12D2B4E7B3, A1E11A12D1, A1F1G12A7, A1H3C5H8E12, B1G1F5A3, and C1B3B12D2 were internalized at relatively high rates.

### Example 6 Generation and Characterization of Chimeric Antibodies

The anti-TROP2 mouse mAbs were sequenced, and the sequence ID numbers of heavy and light chain variable regions were summarized in Table 1.

The variable regions of the heavy and light chains of the anti-TROP2 mouse mAbs A1E4F7D4, A1F1G12A7 and C1B3B12D2 were cloned in frame to human IgG1 heavy-chain (SEQ ID NO.: 64, X1=K, X2=D, X3=L) and human kappa light-chain constant regions (SEQ ID NO.: 65), respectively, wherein the C terminus of the variable region was linked to the N terminus of the respective constant region.

The vectors each containing a nucleotide encoding a heavy chain variable region linked to human IgG1 heavy-chain constant region, and the vectors each containing a nucleotide encoding a light chain variable region linked to human kappa light-chain constant region were transiently transfected into 50 ml of 293F suspension cell cultures in a ratio of 1.1:1 light to heavy chain construct, with 1 mg/mL PEI.

Cell supernatants were harvested after six days in shaking flasks, spun down to pellet cells, and then chimeric antibodies were purified from cell supernatants as described above. The purified antibodies were tested in the capture ELISA, Indirect ELISA, cell based binding FACS, BIAcore affinity test, epitope binning, and cell-based internalization assays following the protocols in the foregoing Examples, with or without minor modifications, as well as protocols described below.

For the BIAcore, goat anti-human IgG (GE healthcare. Cat #BR100839, Human Antibody Capture Kit) was covalently linked to a CM5 chip instead of goat anti-mouse IgG, and a CM5 chip was used for the benchmark instead of a Protein G chip. The results were shown in Table 3.

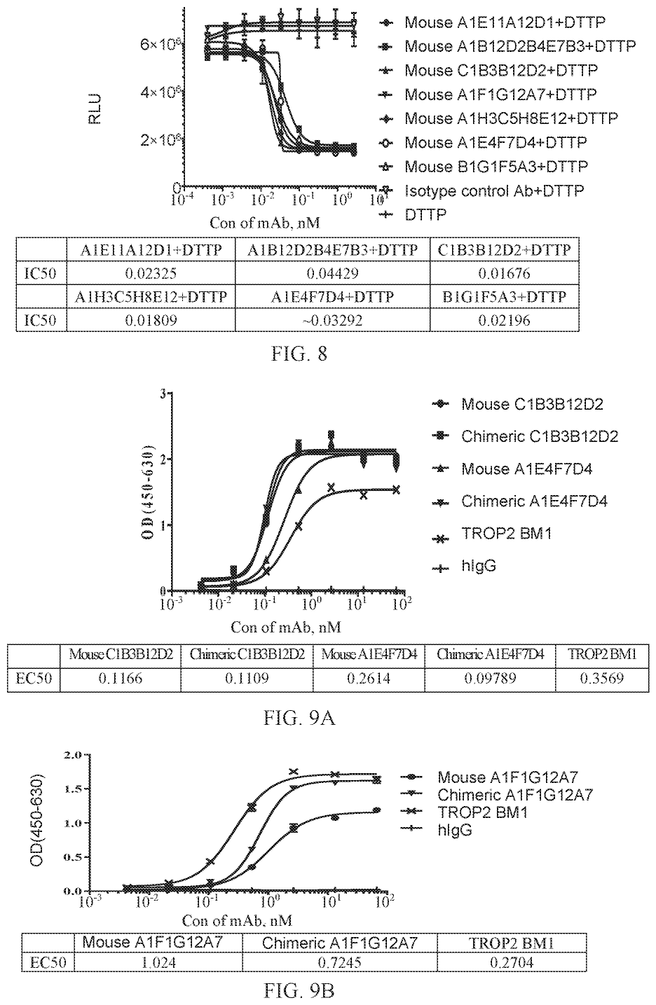

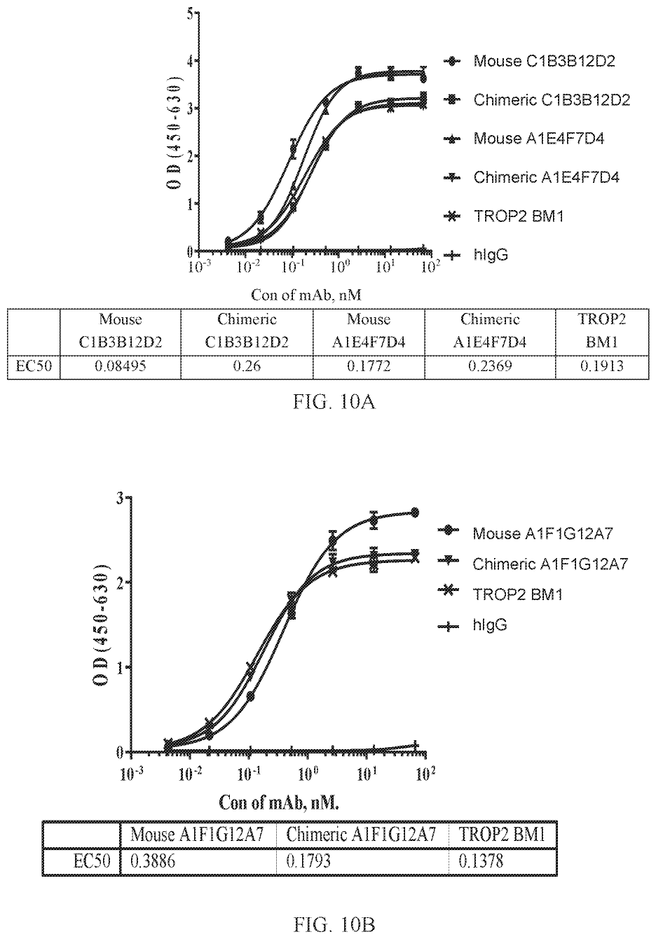

For the capture ELISA, AffiniPure Goat Anti-Human IgG, Fcγ fragment specific (Jackson Immuno Research, Cat #109-005-098) was used instead of AffiniPure Goat Anti-Mouse IgG, Fcγ fragment specific, 100 μl/well. The results were shown in FIGS. 9A-9B.

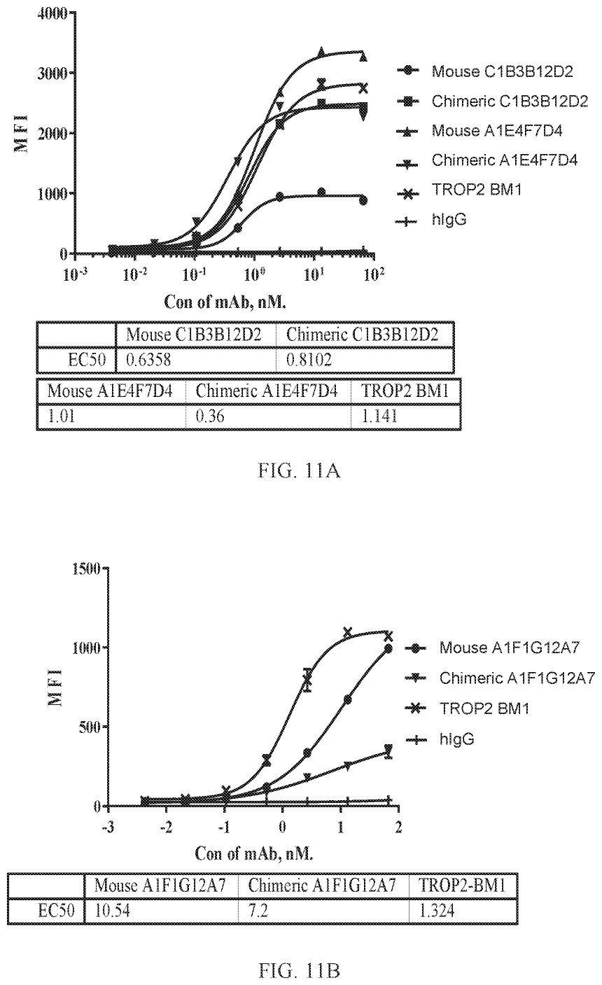

For the indirect ELISA, Peroxidase AffiniPure F(ab′)2 Fragment Goat Anti-Human IgG, Fcγ fragment specific (Jackson Immunoresearch, Cat #109-036-098) was used instead of Peroxidase AffiniPure Goat Anti-Mouse IgG, Fcγ fragment specific, 100 μl/well. The results were shown in FIGS. 10A-10B.

In the cell-based binding FACS, R-Phycoerythrin AffiniPure Goat Anti-Human IgG, Fcγ fragment specific, Jackson Immunoresearch. Cat #109-115-098) was used instead of R-Phycoerythrin AffiniPure F(ab′)₂ Fragment Goat Anti-Mouse IgG (H+L), 100 μl/well. The results were shown in FIGS. 11A-11B.

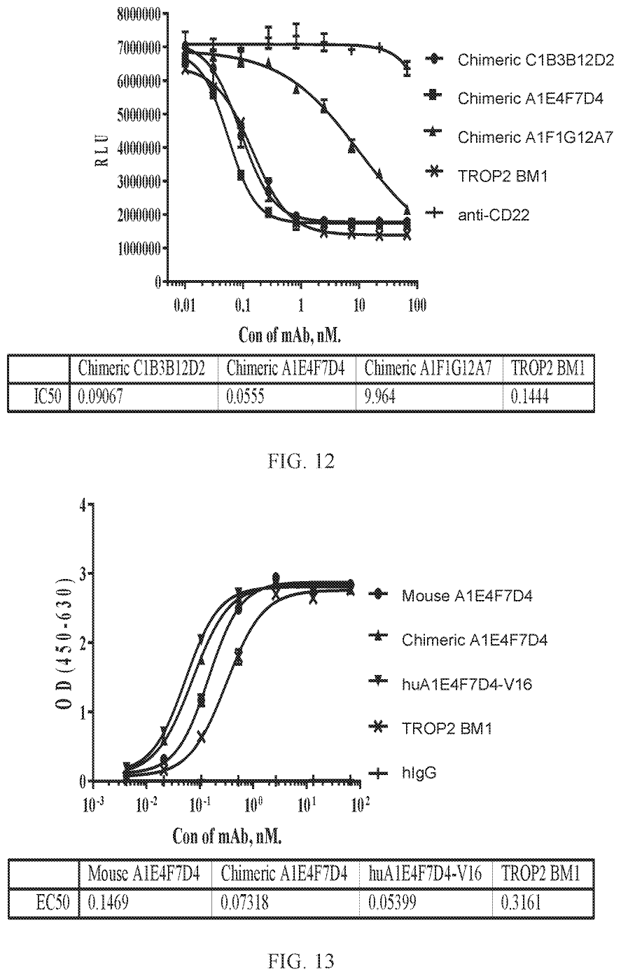

In the cell based internalization assay, a recombinant protein termed DT3C with the amino acid sequence of SEQ ID NO: 73, consisting of diphtheria toxin (DT) lacking the receptor-binding domain and the C1, C2, and C3 domains of Streptococcus protein G (3C), was used to conjugate the antibodies instead of DTTP1170. And an in house made anti-CD22 antibody was used as a negative control. On the next day of cell seeding, the chimeric anti-TROP2 antibodies of the disclosure or controls, 40 μg/mL in FreeStyle293 medium with 10% v/v FBS, were mixed with DT3C protein, 40 μg/mL in FreeStyle293 medium with 10% v/v FBS, at 1:1 volume ratio, and incubated at room temperature for 30 minutes, which were then serially diluted in the cell culture medium, 3-fold serial dilution, starting from 20 μg/mL. Then, 100 μl of the serially diluted antibody/DT3C mixtures were added to the cell plates, and incubated in a CO₂ incubator at 37° C. for 72 hours. The results were shown in FIG. 12.

TABLE 3
Binding Affinity of Chimeric Anti-TROP2 Antibodies to Human TROP2 and Cynomolgus
TROP2

| | Kinetics on Biacore | | | | | |
|---|---|---|---|---|---|---|
| | Human TROP2 | | | cynomolgus TROP2 | | |
| | Kₐ | K_{d} | K_{D} | Kₐ | K_{d} | K_{D} |
| Clone ID | (M−1s−1) | (s−1) | (M) | (M−1s−1) | (s−1) | (M) |
| Mouse A1E4F7D4 | 8.49E+05 | 4.70E−05 | 5.54E−11 | 9.30E+05 | 4.54E−05 | 4.89E−11 |
| Chimeric A1E4F7D4 | 9.61E+05 | 3.13E−05 | 3.26E−11 | 6.92E+05 | 1.71E−04 | 2.47E−10 |
| Mouse A1F1G12A7 | * | * | * | * | * | * |
| Chimeric A1F1G12A7 | 4.50E+05 | 2.45E−04 | 5.45E−10 | * | * | * |
| Mouse C1B3B12D2 | * | * | * | * | * | * |
| Chimeric C1B3B12D2 | 7.63E+05 | 9.54E−05 | 1.25E−10 | * | * | * |
| BM1 | 3.15E+05 | 1.21E−04 | 3.85E−10 | * | * | * |

*Not tested

It can be seen from FIGS. 9A-9B and 11A-11B that the chimeric A1E4F7D4 and C1B3B12D2 antibodies showed higher binding capability than the benchmark in the capture ELISA and/or the cell-based binding FACS test, while the chimeric A1F1G12A7 antibody had a bit lower binding capability than the benchmark in the capture ELISA and the cell-based binding FACS test.

According to FIGS. 10A-10B, the chimeric A1E4F7D4, A1F1G12A7 and C1B3B12D2 antibodies specifically bound the monkey TROP2 protein with comparable binding activity to the benchmark.

FIG. 12 showed that the DT3C conjugates of chimeric A1E4F7D4 and chimeric C1B3B12D2 antibodies were internalized at similar or higher rates compared to benchmark-DT3C conjugate which is now used in clinics. Specifically, the chimeric A1E4F7D4-DT3C conjugates were more efficiently internalized by the target cells, causing target cell death in a more efficacious manner. While the internalization rate of the chimeric A1F1G12A7-DT3C conjugates was much lower than the benchmark-DT3C conjugates.

As summarized in Table 3, the binding affinity of the chimeric antibodies A1E4F7D4 and C1B3B12D2 as tested in the BIAcore test were higher than that of the benchmark.

### Example 7 Humanization of Anti-TROP2 Antibody A1E4F7D4

The mouse anti-TROP2 antibody A1E4F7D4 was humanized and further characterized. Humanization of the antibody was conducted using the well-established CDR-grafting method as described in detail below.

Briefly, the light and heavy chain variable region sequences of the mouse or chimeric antibody A1E4F7D4 were blasted against the human immunoglobulin gene database. The human germlines with the highest homology were selected, and the frameworks from these germlines were used to replace those of the antibody A1E4F7D4. In specific, A1E4F7D4's CDRs were inserted into the selected frameworks, and the residue(s) in the frameworks was/were further back-mutated to obtain more candidate heavy chain/light chain variable regions. A total of 21 exemplary humanized A1E4F7D4 antibodies, namely huA1E4F7D4-V1 to huA1E4F7D4-V21 were obtained whose heavy/light chain variable region sequence ID numbers were in Table 1.

The vectors each containing a nucleotide encoding the heavy chain variable region of one of huA1E4F7D4-V1 to huA1E4F7D4-V21 linked to human IgG1 heavy-chain constant region (SEQ ID NO: 64, X1=K, X2=D, X3=L), and the vectors each containing a nucleotide encoding a humanized light chain variable region linked to human kappa light-chain constant region (SEQ ID NO: 65) were transiently transfected into 50 ml of 293F suspension cell cultures in a ratio of 1.1:1 light to heavy chain construct, with 1 mg/mL PEI.

### Example 8 Characterization of Exemplary Humanized Antibodies

Cell supernatants containing humanized antibodies huA1E4F7D4-V1 to huA1E4F7D4-V21 were harvested after six days in shaking flasks and tested for binding affinity to human TROP2 by Biacore T200 system (GE healthcare, Pittsburgh, PA, USA) following the protocol in the foregoing Example with minor modifications.

The goat anti-human IgG (GE healthcare, Cat #BR100839, Human Antibody Capture Kit) was covalently linked to a CM5 chip instead of goat anti-mouse IgG. Cell supernatants containing humanized antibodies huA1E4F7D4-V1 to huA1E4F7D4-V21 were used instead of purified antibodies. The human TROP2-his protein at the concentration of 40 nM was used instead of serially diluted human TROP2-his protein. The Kₐ, K_{d} and K_{D} values were determined and summarized in Table 4.

The data indicated that the humanized antibodies as tested had high human TROP2 binding affinity.

The humanized antibody huA1E4F7D4-V16 was purified as described above and tested in Biacore, Capture ELISA, Indirect ELISA. Cell-based binding FACS, Competitive ELISA, Cell-based functional assay and Protein thermal shift assay, following the protocols of the foregoing Examples with minor modifications as well as protocols described below.

For the BIAcore, goat anti-human IgG (GE healthcare, Cat #BR100839, Human Antibody Capture Kit) was covalently linked to a CM5 chip instead of goat anti-mouse IgG, and a CM5 chip was used for the benchmark instead of a Protein G chip. The results were shown in Table 6.

For the Capture ELISA, AffiniPure F(ab′)₂ Fragment Goat Anti-Human IgG, Fcγ fragment specific (Jackson Immunoresearch, Cat #109-006-008) was used instead of AffiniPure Goat Anti-Mouse IgG, Fcγ fragment specific, 100 μl/well. The results were shown in FIG. 13.

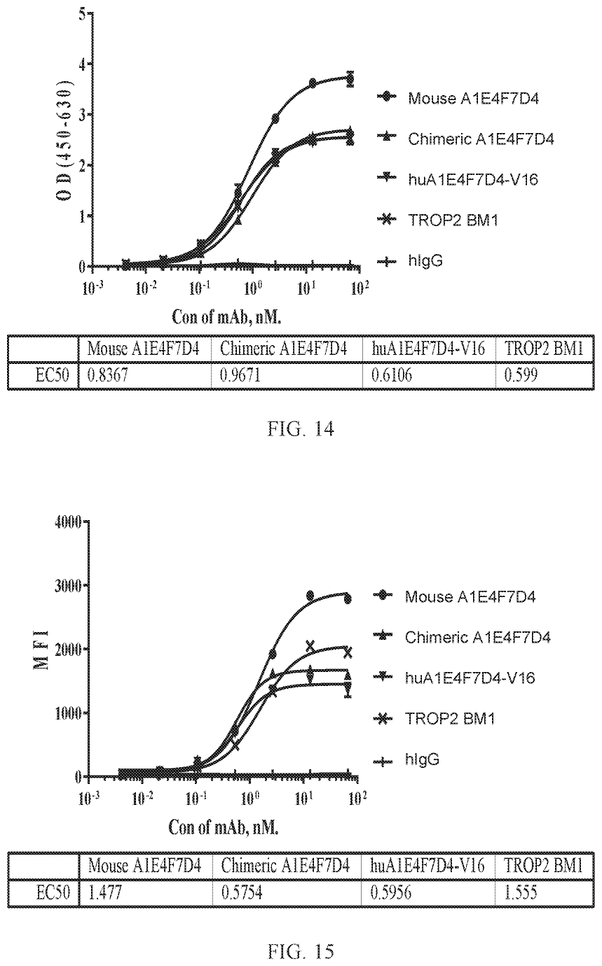

For the Indirect ELISA. Peroxidase AffiniPure F(ab′)₂ Fragment Goat Anti-Human IgG, Fcγ fragment specific (Jackson Immunoresearch, Cat #109-036-098) was used instead of Peroxidase AffiniPure Goat Anti-Mouse IgG, Fcγ fragment specific, 100 μl/well. The results were shown in FIG. 14.

TABLE 4
Binding Affinity of humanized A1E4F7D4 mAbs

| | Kinetics on BIAcore | | |
|---|---|---|---|
| Clone ID | Kₐ (1/Ms) | K_{d} (s−1) | K_{D} (M) |
| huA1E4F7D4-V1 | 8.30E+05 | 1.97E−04 | 2.38E−10 |
| huA1E4F7D4-V2 | 8.74E+05 | 1.88E−04 | 2.15E−10 |
| huA1E4F7D4-V3 | 8.36E+05 | 1.99E−04 | 2.38E−10 |
| huA1E4F7D4-V4 | 8.83E+05 | 1.90E−04 | 2.15E−10 |
| huA1E4F7D4-V5 | 5.77E+05 | 2.04E−04 | 3.53E−10 |
| huA1E4F7D4-V6 | 1.08E+06 | 1.79E−04 | 1.66E−10 |
| huA1E4F7D4-V7 | 8.87E+05 | 1.92E−04 | 2.16E−10 |
| huA1E4F7D4-V8 | 8.32E+05 | 1.95E−04 | 2.35E−10 |
| huA1E4F7D4-V9 | 8.65E+05 | 1.93E−04 | 2.23E−10 |
| huA1E4F7D4-V10 | 6.56E+05 | 2.06E−04 | 3.14E−10 |
| huA1E4F7D4-V11 | 1.08E+06 | 1.75E−04 | 1.63E−10 |
| huA1E4F7D4-V12 | 9.09E+05 | 1.92E−04 | 2.11E−10 |
| huA1E4F7D4-V13 | 8.98E+05 | 1.99E−04 | 2.21E−10 |
| huA1E4F7D4-V14 | 8.34E+05 | 1.93E−04 | 2.32E−10 |
| huA1E4F7D4-V15 | 5.71E+05 | 2.05E−04 | 3.59E−10 |
| huA1E4F7D4-V16 | 1.28E+06 | 1.75E−04 | 1.37E−10 |
| huA1E4F7D4-V17 | 1.20E+06 | 2.79E−04 | 2.32E−10 |
| huA1E4F7D4-V18 | 1.22E+06 | 2.80E−04 | 2.30E−10 |
| huA1E4F7D4-V19 | 1.16E+06 | 3.04E−04 | 2.61E−10 |
| huA1E4F7D4-V20 | 8.68E+05 | 2.90E−04 | 3.35E−10 |
| huA1E4F7D4-V21 | 1.44E+06 | 2.71E−04 | 1.88E−10 |

In the cell-based binding FACS, R-Phycoerythrin AffiniPure Goat Anti-Human IgG, Fcγ fragment specific, Jackson Immunoresearch, Cat #109-115-098) was used instead of R-Phycoerythrin AffiniPure F(ab′)₂ Fragment Goat Anti-Mouse IgG (H+L), 100 μl/well. The results were shown in FIG. 15.

In the cell based internalization assay, the DT3C protein with the amino acid sequence of SEQ ID NO: 73 was used to conjugate the antibodies. On the next day of cell seeding, the anti-TROP2 antibodies of the disclosure or controls, 4.44 sg/mL in FreeStyle293 medium with 10% v/v FBS, were mixed with the DT3C protein, 4.44 μg/mL in FreeStyle293 medium with 10% v/v FBS, at 1:1 volume ratio, and incubated at room temperature for 30 minutes, which were then serially diluted in the cell culture medium, 3-fold serial dilution, starting from 2.22 μg/mL. Then, 100 μl of the serially diluted antibody/DT3C mixtures were added to the cell plates, and incubated in a CO₂ incubator at 37° C. for 72 hours. The results were shown in FIG. 17.

For the thermal shift assay, a protein thermal shift assay was used to determine Tm (melting temperature) using a GloMelt™ Thermal Shift Protein Stability Kit (Biotium, Cat #33022-T). Briefly, the GloMelt™ dye was allowed to thaw and reach room temperature. The vial containing the dye was vortexed and centrifuged. Then, 10×dye was prepared by adding 5 μL 200×dye to 95 μL PBS, 2 μL 10× dye and 10 μg humanized antibodies were added, and PBS was added to a total reaction volume of 20 μL. The tubes containing the dye and antibodies were briefly spun and placed in real-time PCR thermocycler (Roche, LightCycler 480 II) set up with a melt curve program having the parameters in Table 5. The results were shown in FIG. 18.

TABLE 5
Parameters for Melt Curve Program

| Profile step | Temperature | | Ramp rate | Holding Time |
|---|---|---|---|---|
| Initial hold | 25° | C. | NA | 30 s |
| Melt curve | 25-99° | C. | 0.1° C./s | NA |

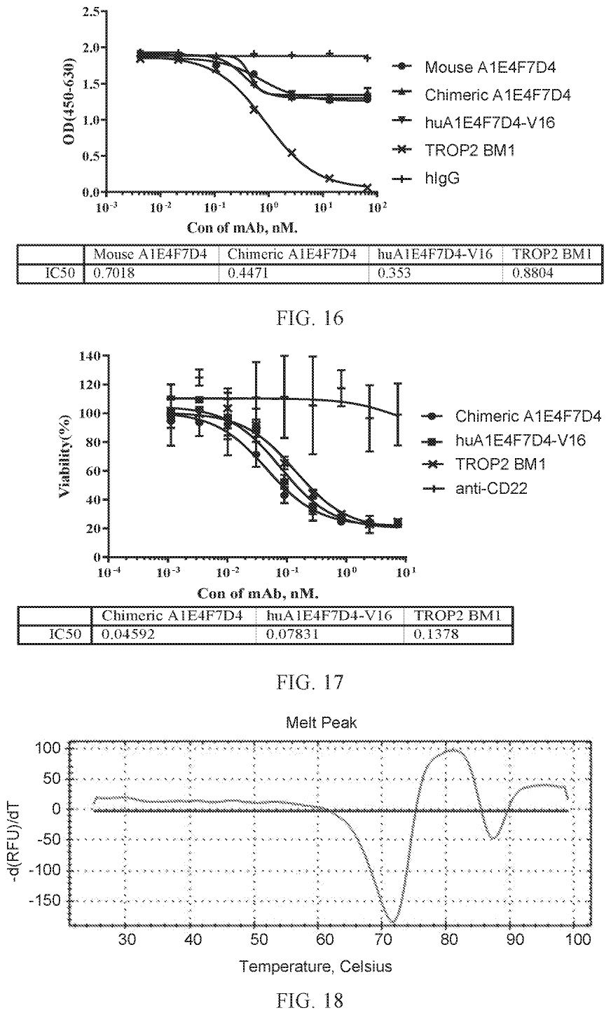

Results of the huA1E4F7D4-V16's blocking activity on benchmark-human TROP2 binding were shown in FIG. 16.

TABLE 6
Binding affinity of humanized mAbs

| | Kinetics on Biacore | | | | | |
|---|---|---|---|---|---|---|
| | Human TROP2-his | | | Cynomolgus TROP2-his | | |
| | Kₐ | K_{d} | K_{D} | Kₐ | K_{d} | K_{D} |
| Clone ID# | (M⁻¹s⁻¹) | (s⁻¹) | (M) | (M⁻¹s⁻¹) | (s⁻¹) | (M) |
| chimeric A1E4F7D4 | 1.05E+06 | 1.94E−04 | 1.85E−10 | 1.22E+06 | 1.63E−04 | 1.34E−10 |
| huA1E4F7D4-V16 | 1.06E+06 | 1.95E−04 | 1.83E−10 | 1.25E+06 | 1.64E−04 | 1.32E−10 |
| BM1 | 2.83E+05 | 2.78E−04 | 9.83E−10 | 4.77E+05 | 2.53E−04 | 5.29E−10 |

According to Table 6, the antibody huA1E4F7D4-V16 showed comparable binding affinity to human and monkey TROP2 proteins as compared to the chimeric A1E4F7D4 antibody, which was higher than that of the benchmark.

It can be seen from FIGS. 13 and 15 that the humanized antibody huA1E4F7D4-V16 specifically bound to human TROP2 with a lower EC₅₀ than the benchmark, suggesting that it more efficiently bound to the human TROP2 protein. As shown in FIG. 14, huA1E4F7D4-V16 bound monkey TROP2 at a comparable activity compared to the benchmark.

As shown in FIG. 16, the humanized antibody huA1E4F7D4-V16 did not block benchmark (TROP2 BM1) binding to human TROP2, suggesting that this antibody might bind to a different epitope as compared to the benchmark (TROP2 BM1).

FIG. 17 showed that huA1E4F7D4-V16-DT3C conjugates were internalized at a higher rate than the benchmark-DT3C conjugates, meaning that huA1E4F7D4-V16-DT3C conjugates were more efficiently internalized by the target cells, causing target cell death in a more efficacious manner.

Further, as shown in FIG. 18, the melting temperatures of huA1E4F7D4-V16 were 71.5° C. and 87.5° C.

### Example 9 Characterization of Humanized Antibody huA1E4F7D4-V16

The humanized antibody huA1E4F7D4-V16 was tested in Biacore, Cell-based binding FACS, Cell based internalization assay and Epitope grouping ELISA, following the protocols of the foregoing Examples, with or without minor modifications, as well as the protocols described below, in comparison to an analog of Datopotamab (Daiichi Sankyo's anti-trop2 mAb, Dato-DXd, DS-1062a), also referred to as BM2, which was in house made with the heavy and light chain amino acid sequences of SEQ ID NOs: 76 and 77, respectively.

For the BIAcore, the results were shown in Table 7.

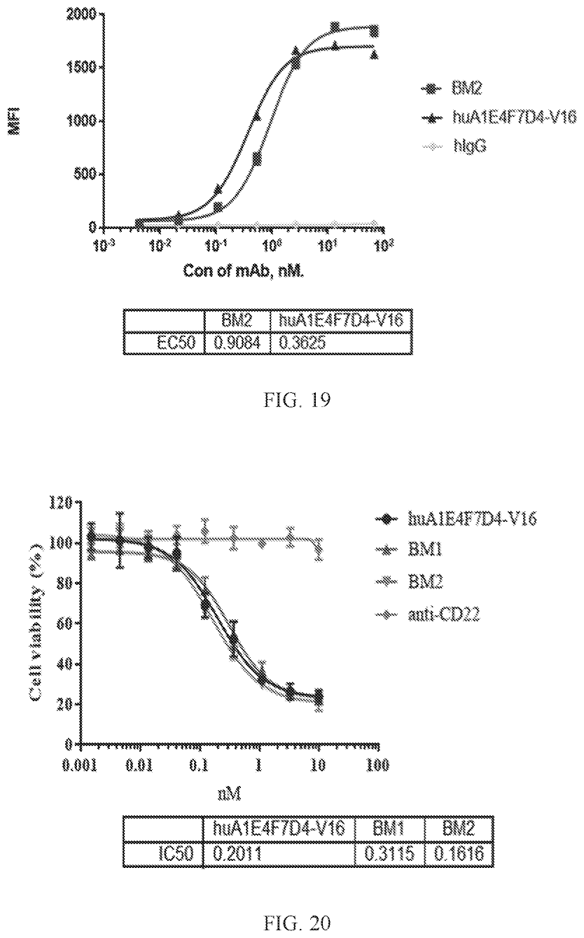

For the cell-based binding FACS, the results were shown in FIG. 19.

In the cell based internalization assay, the DT3C protein with the amino acid sequence of SEQ ID NO: 73 was used to conjugate the antibodies. On the next day of cell seeding, huA1E4F7D4-V16 or controls, 4.44 μg/mL in FreeStyle293 medium with 10% v/v FBS, were mixed with the DT3C protein, 4.44 μg/mL in FreeStyle293 medium with 10% v/v FBS, at 1:1 volume ratio, and incubated at room temperature for 30 minutes, which were then serially diluted in the cell culture medium, 3-fold serial dilution, starting from 2.22 μg/mL. Then, 100 μl of the serially diluted antibody/DT3C mixtures were added to the cell plates, and incubated in a CO₂ incubator at 37° C. for 72 hours. The results were shown in FIG. 20.

TABLE 7
Binding affinity of huA1E4F7D4-V16

| | Kinetics on Biacore | | |
|---|---|---|---|
| | Human TROP2-his | | |
| | Kₐ | K_{d} | K_{D} |
| Clone ID# | (M⁻¹s⁻¹) | (s⁻¹) | (M) |
| huA1E4F7D4-V16 | 4.04E+05 | 7.92E−05 | 1.96E−10 |
| BM2 | 1.27E+05 | 3.64E−03 | 2.87E−08 |

The results showed that huA1E4F7D4-V16 had over 100-fold higher affinity to human TROP2 and better cell binding ability than BM2, and comparable internalization rate to BM2.

### Epitope Binning

Epitope binning ELISA was performed to determine whether the epitope bound by huA1E4F7D4-V16 and that by BM1 or BM2 overlap to some extent.

Firstly, capture ELISA was performed to determine the concentration of biotin-labeled human Trop2 proteins appropriate for the epitope binning test. Briefly, 96-well plates were coated with 2 μg/ml huA1E4F7D4-V16, BM1 or BM2 in PBS, respectively, 100 μl/well, overnight at 4′C, and blocked by 5% non-fatty milk in PBST for 2 hours at 37° C. The plates were washed for 4 times, added with 100 μl/well of serially diluted biotin human Trop2-his proteins (SEQ ID NO: 67) in PBST with 2.5% non-fatty milk (starting from 1.3 μg/ml with a 5-fold serial dilution), and incubated for 40 min at 37° C. Then the plates were washed for 4 times and added with 100 μl/vell of HRP-streptavidin (Jackson Immuno Research, Cat #016-030-084). The plates were incubated for another 40 min at 37° C. Then the plates were washed again, and 100 μl/well TMB was added for color development at RT for 15 min followed by quenching with 50 μl 1M H₂SO₄. The OD values at 450 nm were read. The concentration at which the antibody gave an OD₄₅₀ value around 2.0 was picked for the epitope binning test.

With the appropriate concentration determined above, epitope grouping ELISA was performed. Briefly, 100 μl of BM1 at 2 μg/mL, BM2 at 2 μg/mL, and huA1E4F7D4-V16 at 2 μg/mL, in PBS were respectively coated on 96-well micro plates for 2 hours at 37° C. ELISA plates were washed once with wash buffer (PBS+0.05% v/v Tween-20, PBST) and then blocked with 200 μl blocking buffer (5% w/w non-fatty milk in PBST) for 2 hours at 37° C. While blocking, huA1E4F7D4-V16, BM1 and BM2 were respectively mixed with the human biotin-human Trop2 proteins, wherein in the mixtures huA1E4F7D4-V16, BM1 and BM2 were at the final concentration of 15 μg/ml and the human biotin-human Trop2 proteins were at the final concentration determined above. The mixtures were incubated at room temperature for 40 minutes. After plate washing for 4 times, the antibody/biotin-TROP2-his protein mixtures were added to the antibody coated plates, 100 μl per well, and incubated for another 40 min at 37° C. Then 100 μl/well of HRP-streptavidin was added and incubated for 40 min. Then OD₄₅₀ values were determined and the cross-competition capability was calculated (cross-competition capability of antibody X (%)=(OD₄₅₀ of Antibody X-OD₄₃₀ of Blank)/(OD₄₅₀ of no mAb-OD₄₃₀ of Blank)*100%).

The antibodies were considered to bind the same epitope when their cross-competition capability was higher than 80%.

The results were shown in Table 8 below. It can be seen that huA1E4F7D4-V16 did not block benchmark binding to human TROP2, suggesting that it might bind to a different epitope as compared to the BM1 and BM2.

TABLE 8
Epitope binning results
OD₄₅₀

| | BM2-coated | BMI-coated | huA1E4F7D4-V16- |
|---|---|---|---|
| | plate | plate | coated plate |
| BM2 | 0.354 | 0.519 | 1.629 |
| BM1 | 0.031 | 0.048 | 1.614 |
| huA1E4F7D4-V16 | 1.919 | 1.424 | 0.027 |
| no mAb | 2.666 | 2.15 | 1.853 |
| Blank | 0.014 | 0.017 | 0.016 |

While the disclosure has been described above in connection with one or more embodiments, it should be understood that the disclosure is not limited to those embodiments, and the description is intended to cover all alternatives, modifications, and equivalents, as may be included within the spirit and scope of the appended claims. All referenced cited herein are further incorporated by reference in their entirety.

Sequences in the present application are summarized below.

Description/
Sequence/SEQ ID NO.

VH CDR1 of mouse, chimeric and humanized A1E4F7D4
THWIH
(SEQ ID NO: 1)
VH CDR2 of mouse, chimeric and humanized A1E4F7D4
TIFPSNAYAVYNQKFRD
(SEQ ID NO: 2)
VH CDR3 of mouse, chimeric and humanized A1E4F7D4
ASYFDY
(SEQ ID NO: 3)
VL CDR1 of mouse, chimeric and humanized A1E4F7D4
RASQNIGTSIH
(SEQ ID NO: 4)
VL CDR2 of mouse, chimeric and humanized A1E4F7D4
YASESIS
(SEQ ID NO: 5)
VL CDR3 of mouse, chimeric and humanized A1E4F7D4
QHSHSWPFT
(SEQ ID NO: 6)
VH of mouse and chimeric A1E4F7D4
QVQLQQSGAELAKPGTSVKMSCEASGYSFTTHWIHWMKQRPGQGL
EWIGTIFPSNAYAVYNQKFRDRAIMTADRSSTTAYIQLTGLTSED
SAVYYCARASYFDYWGQGTTLTVSS
(SEQ ID NO: 44)
CAGGTCCAGCTGCAGCAGTCTGGGGCTGAGCTGGCAAAACCTGGG
ACCTCAGTGAAGATGTCCTGCGAGGCTTCTGGCTACTCCTTTACT
ACCCACTGGATACACTGGATGAAGCAGAGGCCTGGACAGGGTCTG
GAATGGATTGGGACTATTTTTCCTAGCAATGCTTATGCTGTTTAT
AATCAGAAATTCAGGGACAGGGCCATAATGACTGCAGACAGATCC
TCCACTACAGCCTATATACAACTCACCGGCCTGACATCTGAGGAC
TCTGCAGTCTATTACTGTGCAAGAGCCAGTTACTTTGACTACTGG
GGCCAAGGCACCACTCTCACAGTCTCCTCA
(SEQ ID NO: 41)
VH of huA1E4F7D4-V1
QVQLVQSGAEVKKPGASVKVSCKASGYSFTTHWIHWMKQRPGQGL
EWIGTIFPSNAYAVYNQKFRDRAIMTADRSISTAYIELTRLRSDD
TAVYYCARASYFDYWGQGTTVTVSS
(SEQ ID NO: 45)
VH of huA1E4F7D4-V2, huA1E4F7D4-V7,
huA1E4F7D4-V12 and huA2G10B1C2-V17
QVQLVQSGAEVKKPGASVKVSCKASGYX1FTTHWIHWVRQAPGQG
LEWIGTIFPSNAYAVYNQKFRDRX2TMTADRSISTAYIELSRLRS
DDTAVYYCARASYFDYWGQGTTVTVSS
(SEQ ID NO: 46) X1 = S, X2 = A
QVQLVQSGAEVKKPGASVKVSCKASGYSFTTHWIHWVRQAPGQGL
EWIGTIFPSNAYAVYNQKFRDRATMTADRSISTAYIELSRLRSDD
TAVYYCARASYFDYWGQGTTVTVSS
VH of huA1E4F7D4-V3, huA1E4F7D4-V8,
huA1E4F7D4-V13 and huA1E4F7D4-V18
QVQLVQSGAEVKKPGASVKVSCKASGYX1FTTHWIHWVRQAPGQG
LEWIGTIFPSNAYAVYNQKFRDRX2TMTADRSISTAYIELSRLRS
DDTAVYYCARASYFDYWGQGTTVTVSS
(SEQ ID NO: 46) X1 = T, X2 = A
QVQLVQSGAEVKKPGASVKVSCKASGYTFTTHWIHWVRQAPGQGL
EWIGTIFPSNAYAVYNQKFRDRATMTADRSISTAYIELSRLRSDD
TAVYYCARASYFDYWGQGTTVTVSS
VH of huA1E4F7D4-V4, huA1E4F7D4-V9,
huA1E4F7D4-V14 and huA1E4F7D4-V19
QVQLVQSGAEVKKPGASVKVSCKASGYX1FTTHWIHWVRQAPGQG
LEWIGTIFPSNAYAVYNQKFRDRX2TMTADRSISTAYIELSRLRS
DDTAVYYCARASYFDYWGQGTTVTVSS
(SEQ ID NO: 46) X1 = S, X2 = V
QVQLVQSGAEVKKPGASVKVSCKASGYSFTTHWIHWVRQAPGQGL
EWIGTIFPSNAYAVYNQKFRDRVTMTADRSISTAYIELSRLRSDD
TAVYYCARASYFDYWGQGTTVTVSS
VH of huA1E4F7D4-V5, huA1E4F7D4-V10,
huA1E4F7D4-V15 and huA1E4F7D4-V20
QVQLVQSGAEVKKPGASVKVSCKASGYSFTTHWIHWVRQAPGQGL
EWIGTIFPSNAYAVYNQKFRDRATMTX1DX2SISTAYIELSRLRS
DDTAVYYCARASYFDYWGQGTTVTVSS
(SEQ ID NO: 47) X1 = R, X2 = R
QVQLVQSGAEVKKPGASVKVSCKASGYSFTTHWIHWVRQAPGQGL
EWIGTIFPSNAYAVYNQKFRDRATMTRDRSISTAYIELSRLRSDD
TAVYYCARASYFDYWGQGTTVTVSS
VH of huA1E4F7D4-V6, huA1E4F7D4-V11,
huA1E4F7D4-V16 and huA1E4F7D4-V21
QVQLVQSGAEVKKPGASVKVSCKASGYSFTTHWIHWVRQAPGQGL
EWIGTIFPSNAYAVYNQKFRDRATMTX1DX2SISTAYIELSRLRS
DDTAVYYCARASYFDYWGQGTTVTVSS
(SEQ ID NO: 47) X1 = A, X2 = T
QVQLVQSGAEVKKPGASVKVSCKASGYSFTTHWIHWVRQAPGQGL
EWIGTIFPSNAYAVYNQKFRDRATMTADTSISTAYIELSRLRSDD
TAVYYCARASYFDYWGQGTTVTVSS
CAAGTGCAGCTGGTGCAGTCTGGGGCTGAGGTGAAGAAGCCTGGG
GCCTCTGTGAAGGTGAGCTGCAAGGCCTCTGGCTACAGCTTCACC
ACCCACTGGATCCACTGGGTGAGACAAGCCCCTGGCCAAGGCCTG
GAGTGGATTGGCACCATCTTCCCTAGCAATGCCTATGCTGTGTAC
AATCAGAAGTTCAGAGACAGAGCCACCATGACAGCTGACACAAGC
ATCAGCACAGCCTACATTGAGCTGAGCAGACTGAGATCTGATGAC
ACAGCTGTGTACTACTGTGCTAGAGCTAGCTACTTTGACTACTGG
GGCCAAGGCACCACAGTGACAGTGAGCAGC
(SEQ ID NO: 42)
VL of mouse and chimeric A1E4F7D4
DILLTQSPATLSVSPGERVTFSCRASQNIGTSIHWFQQRTTGSPR
LLIKYASESISGIPSRFSGSGSGTDFTLHINSVESEDIAHYYCQH
SHSWPFTFGSGTKLEIQ
(SEQ ID NO: 48)
GACATCTTGTTGACTCAGTCTCCAGCCACCCTGTCTGTGAGTCCA
GGAGAAAGAGTCACTTTCTCCTGCAGGGCCAGTCAGAACATTGGC
ACAAGCATACACTGGTTTCAGCAAAGAACAACTGGTTCTCCAAGG
CTTCTCATAAAGTATGCTTCTGAGTCTATCTCTGGGATCCCTTCC
AGATTTAGTGGCAGTGGATCAGGGACAGATTTTACTCTTCACATC
AACAGTGTGGAGTCTGAAGATATTGCACATTATTACTGTCAACAT
AGTCATAGCTGGCCATTCACGTTCGGCTCGGGGACAAAGTTGGAA
ATACAA
(SEQ ID NO: 43)
VL of huA1E4F7D4-V1
X1IX2LTQSPATLSLSPGERATLSCRASQNIGTSIHWFQQKPGGS
PRLLIKYASESISGIPSRFSGSGSGTDFTLTISSX3EPEDFAVYY
CQHSHSWPFTFGQGTKLEIK
(SEQ ID NO: 49) X1 = D,
X2 = L, X3 = V
DILLTQSPATLSLSPGERATLSCRASQNIGTSIHWFQQKPGGSPR
LLIKYASESISGIPSRFSGSGSGTDFTLTISSVEPEDFAVYYCQH
SHSWPFTFGQGTKLEIK
VL of huA1E4F7D4-V2-huA1E4F7D4-V6
X1IX2LTQSPATLSLSPGERATLSCRASQNIGTSIHWFQQKPGGS
PRLLIKYASESISGIPSRFSGSGSGTDFTLTISSX3EPEDFAVYY
CQHSHSWPFTFGQGTKLEIK
(SEQ ID NO: 49) X1 = E,
X2 = V, X3 = L
EIVLTQSPATLSLSPGERATLSCRASQNIGTSIHWFQQKPGGSPR
LLIKYASESISGIPSRFSGSGSGTDFTLTISSLEPEDFAVYYCQH
SHSWPFTFGQGTKLEIK
VL of huA1E4F7D4-V7-huA1E4F7D4-V11
EIVLTQSPATLSLSPGERATLSCRASQNIGTSIHWFQQKPGX1X2
PRLLIX3YASESISGIPSRFSGSGSGTDFTLTISSLEPEDFAVYY
CQHSHSWPFTFGQGTKLEIK
(SEQ ID NO: 50) X1 = Q, X2 = S, X3 = K
EIVLTQSPATLSLSPGERATLSCRASQNIGTSIHWFQQKPGQSPR
LLIKYASESISGIPSRFSGSGSGTDFTLTISSLEPEDFAVYYCQH
SHSWPFTFGQGTKLEIK
VL of huA1E4F7D4-V12-huA1E4F7D4-V16
EIVLTQSPATLSLSPGERATLSCRASQNIGTSIHWFQQKPGX1X2
PRLLIX3YASESISGIPSRFSGSGSGTDFTLTISSLEPEDFAVYY
CQHSHSWPFTFGQGTKLEIK
(SEQ ID NO: 50) X1 = G,
X2 = A, X3 = K
EIVLTQSPATLSLSPGERATLSCRASQNIGTSIHWFQQKPGGAPR
LLIKYASESISGIPSRFSGSGSGTDFTLTISSLEPEDFAVYYCQH
SHSWPFTFGQGTKLEIKGAGATTGTGCTGACACAGAGCCCTGCCA
CCCTGAGCCTGTCCCCTGGGGAGAGAGCTACCCTGAGCTGCAGAG
CTTCTCAGAACATTGGCACAAGCATCCACTGGTTTCAGCAGAAGC
CTGGGGGCGCCCCTAGACTGCTGATCAAGTATGCCTCTGAGAGCA
TCTCTGGCATCCCTAGCAGATTCTCTGGCTCTGGCTCTGGCACAG
ACTTCACCCTGACCATCAGCAGCCTGGAGCCTGAGGACTTTGCTG
TGTACTACTGTCAGCACAGCCACAGCTGGCCCTTCACCTTTGGCC
AAGGCACCAAGCTGGAGATCAAG
(SEQ ID NO: 63)
VL of huA1E4F7D4-V17-huA1E4F7D4-V21
EIVLTQSPATLSLSPGERATLSCRASQNIGTSIHWFQQKPGX1X2
PRLLIX3YASESISGIPSRFSGSGSGTDFTLTISSLEPEDFAVYY
CQHSHSWPFTFGQGTKLEIK
(SEQ ID NO: 50) X1 = G,
X2 = S, X3 = Y
EIVLTQSPATLSLSPGERATLSCRASQNIGTSIHWFQQKPGGSPR
LLIYYASESISGIPSRFSGSGSGTDFTLTISSLEPEDFAVYYCQH
SHSWPFTFGQGTKLEIK
VH CDR1 of mouse A1E11A12D1
TYGIS
(SEQ ID NO: 7)
VH CDR2 of mouse A1E11A12D1
QIYPGSDYSYCDEDFKG
(SEQ ID NO: 8)
VH CDR3 of mouse A1E11A12D1
ASYFDY
(SEQ ID NO: 3)
VL CDR1 of mouse A1E11A12D1
RASQTIGTAIH
(SEQ ID NO: 9)
VL CDR2 of mouse A1E11A12DI
YASESIS
(SEQ ID NO: 10)
VL CDR3 of mouse A1E11A12D1
QQSNNWPFT
(SEQ ID NO: 11)
VH of mouse A1E11A12D1
QVQLQQSGAELARPGASVKLSCMASGYTFTTYGISWVRQRTGQGL
EWIGQIYPGSDYSYCDEDFKGKATLTADKSSSTAYMQLSSLTSED
SAVYFCARASYFDYWGQGTTLTVSS
(SEQ ID NO: 51)
VL of mouse A1E11A12D1
DILLTQSPAILSVSPGERVSFSCRASQTIGTAIHWYQQRANGSPR
LLIKYASESISGIPSRFSGSGSGTDFALSINTVESEDFAYYYCQQ
SNNWPFTFGGGTKLEIR
(SEQ ID NO: 52)
VH CDR1 of mouse A1H3C5H8E12
NYWID
(SEQ ID NO: 12)
VH CDR2 of mouse A1H3C5H8E12
NIFPGGFYTNYNEKFKG
(SEQ ID NO: 13)
VH CDR3 of mouse A1H3C5H8E12
GGVFDY
(SEQ ID NO: 14)
VL CDR1 of mouse A1H3C5H8E12
RASQSIGTSIH
(SEQ ID NO: 15)
VL CDR2 of mouse A1H3C5H8E12
YASESIS
(SEQ ID NO: 16)
VL CDR3 of mouse A1H3C5H8E12
QQSNSWPYT
(SEQ ID NO: 17)
VH of mouse A1H3C5H8E12
QVQLQQSGAELVRPGTSVKMSCKAAGYTFTNYWIDWVKQRPGHGL
EWIGNIFPGGFYTNYNEKFKGKATLTADTSSSTAYMQLSSLTSED
SAIYYCARGGVFDYWGQGTTLTVSS
(SEQ ID NO: 53)
VL of mouse A1H3C5H8E12
DILLTQSPAILSVSPGERVSFSCRASQSIGTSIHWYQQRTNGSPR
LLIKYASESISGIPSRESGSGSGTDFTLSINSVESEDIADYYCQQ
SNSWPYTFGGGTKLEIK
(SEQ ID NO: 54)
VH CDR1 of mouse and chimeric A1F1G12A7
SYGVH
(SEQ ID NO: 18)
VH CDR2 of mouse and chimeric A1F1G12A7
VIWRGGITDYNAAFIS
(SEQ ID NO: 19)
VH CDR3 of mouse and chimeric A1F1G12A7
DGDYEYYTMDY
(SEQ ID NO: 20)
VL CDR1 of mouse and chimeric A1F1G12A7
RASKSVSTSGYSYMH
(SEQ ID NO: 21)
VL CDR2 of mouse and chimeric A1F1G12A7
LASDQDS
(SEQ ID NO: 22)
VL CDR3 of mouse and chimeric A1F1G12A7
QHSRELPYT
(SEQ ID NO: 23)
VH of mouse and chimeric A1F1G12A7
QVQLKQSGPGLVQPSQSLSITCTVSGFSLTSYGVHWVRQSPGKGL
EWLGVIWRGGITDYNAAFISRLSISKDNSKSQIFFKMNSLQGDDT
AIYYCARDGDYEYYTMDYWGQGTSVTVSS
(SEQ ID NO: 55)
VL of mouse and chimeric A1F1G12A7
DIVLTQSPASLAVSLGQRATISCRASKSVSTSGYSYMHWNQQKPG
QSPKLLIYLASDQDSGVPARFSGSGSGTDFTLSIHPVEEEDAATY
YCQHSRELPYTFGGGTKLEIK
(SEQ ID NO: 56)
VH CDR1 of mouse A1B12D2B4E7B3
DYYMN
(SEQ ID NO: 24)
VH CDR2 of mouse A1B12D2B4E7B3
YIYPNHGGTGYNQKFKD
(SEQ ID NO: 25)
VH CDR3 of mouse A1B12D2B4E7B3
ENYGYAMDY
(SEQ ID NO: 26)
VL CDR1 of mouse A1B12D2B4E7B3
RSSQSLVHGDGNTYLH
(SEQ ID NO: 27)
VL CDR2 of mouse A1B12D2B4E7B3
TVSNRFS
(SEQ ID NO: 28)
VL CDR3 of mouse A1B12D2B4E7B3
SQTTHVPT
(SEQ ID NO: 29)
VH of mouse A1B12D2B4E7B3
EVQLQQSGPELVKPGASVKMSCKASGFTFTDYYMNWVKQSHGKSL
EWIGYIYPNHGGTGYNQKFKDKATLTVDKSSSTAYMELRSLTSDD
SAVYYCARENYGYAMDYWGQGTSVTVSS
(SEQ ID NO: 57)
VL of mouse A1B12D2B4E7B3
DVVMTQTPLSLPVSLGDQASISCRSSQSLVHGDGNTYLHWFLQKP
GQSPKLLIYTVSNRFSGVPDRFSGSGSGTDFTLKINRVEAEDLGI
YFCSQTTHVPTFGGGTKLEIK
(SEQ ID NO: 58)
VH CDR1 of mouse B1G1F5A3
DYSMN
(SEQ ID NO: 30)
VH CDR2 of mouse B1G1F5A3
YIYPNNGASGFNQKFKG
(SEQ ID NO: 31)
VH CDR3 of mouse B1G1F5A3
EQDNSGYCFDY
(SEQ ID NO: 32)
VL CDR1 of mouse B1G1F5A3
RSSLNLVHSNGNTFLH
(SEQ ID NO: 33)
VL CDR2 of mouse B1G1F5A3
KVSNRFS
(SEQ ID NO: 34)
VL CDR3 of mouse B1G1F5A3
SQTTHVPT
(SEQ ID NO: 29)
VH of mouse B1G1F5A3
EVQLQQSGPELVKPGTSVKMSCKASGYSFADYSMNWVRQSHGNSL
EWIGYIYPNNGASGFNQKFKGKATLTVDKSSSTAYMELHSLTSED
SAVYYCAREQDNSGYCFDYWGQGTSLTVSS
(SEQ ID NO: 59)
VL of mouse B1G1F5A3
DVVMTQTPLSLAVRLGDQASISCRSSLNLVHSNGNTFLHWYLQRP
GQSPKLLIYKVSNRFSGVSDRFSGSGSGTDFTLKISRVEAEDLGV
YFCSQTTHVPTFGGGTKLEIK
(SEQ ID NO: 60)
VH CDR1 of mouse and chimeric C1B3B12D2
KFWIG
(SEQ ID NO: 35)
VH CDR2 of mouse and chimeric C1B3B12D2
NIYPGGAYINYNENFKG
(SEQ ID NO: 36)
VH CDR3 of mouse and chimeric C1B3B12D2
EGSSGY
(SEQ ID NO: 37)
VL CDR1 of mouse and chimeric C1B3B12D2
KCSQSLLNSGTQENYLT
(SEQ ID NO: 38)
VL CDR2 of mouse and chimeric C1B3B12D2
WASIREP
(SEQ ID NO: 39)
VL CDR3 of mouse and chimeric C1B3B12D2
QHDYSYPFT
(SEQ ID NO: 40)
VH of mouse and chimeric C1B3B12D2
QVQLQQSGPELVRPGTSVKMSCKAAGYTFTKFWIGWVKQRPGHGL
EWIGNIYPGGAYINYNENFKGKATLTADTFSSTAYMQLNSLTSAD
SAIYYCAREGSSGYWGQGTTLTVSS
(SEQ ID NO: 61)
VL of mouse and chimeric C1B3B12D2
DIVMTQSPSSLTVTAGEKVTLSCKCSQSLLNSGTQENYLTWYQQK
PGQPPKMLIYWASIREPGVPDRFTGSGSGTDFTLTINNVQAEDLA
VYYCQHDYSYPFTFGSGTKLEIK
(SEQ ID NO: 62)
Heavy chain constant region of chimeric and
humanized IgG antibodies
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKXIVEPKSCDKTHTCPPCPAPELLGGPSVFLFPPKPKDTL
MISRTPEVTCVVVDVSHEDPEVKFNWYVDGVEVHNAKTKPREEQY
NSTYRVVSVLTVLHQDWLNGKEYKCKVSNKALPAPIEKTISKAKG
QPREPQVYTLPPSRX2EX3TKNQVSLTCLVKGFYPSDIAVEWESN
GQPENNYKTTPPVLDSDGSFFLYSKLTVDKSRWQQGNVFSCSVMH
EALHNHYTQKSLSLSPGK
(SEQ ID NO: 64)
SEQ ID NO: 64, X1 = R, X2 = E, X3 = M
SEQ ID NO: 64, X1 = K, X2 = D, X3 = L
GCCTCTACTAAAGGGCCCTCGGTATTCCCGCTGGCACCTTCATCA
AAGTCCACTTCAGGAGGAACAGCAGCACTTGGATGTCTCGTTAAG
GACTATTTCCCAGAACCAGTCACTGTTTCCTGGAATTCAGGAGCA
CTTACATCAGGAGTGCACACATTTCCTGCAGTGCTTCAATCATCA
GGACTTTACTCACTATCCTCGGTAGTCACGGTGCCTTCATCATCA
CTTGGAACACAAACATACATCTGCAACGTGAACCACAAACCTTCG
AATACTAAAGTCGATAAGAAGGTCGAGCCTAAATCATGCGATAAG
ACCCATACATGCCCTCCTTGCCCTGCACCTGAACTTCTTGGAGGG
CCGAGTGTGTTTCTGTTTCCTCCTAAGCCCAAGGATACACTTATG
ATCTCAAGAACACCTGAAGTGACATGCGTGGTGGTGGATGTGTCA
CACGAAGATCCTGAAGTGAAATTTAACTGGTACGTGGATGGAGTG
GAAGTGCACAACGCAAAGACTAAGCCTAGAGAAGAACAATACAAC
TCAACATACAGAGTGGTGTCAGTGCTTACAGTGCTTCACCAAGAT
TGGCTTAACGGAAAGGAGTATAAATGCAAAGTGTCAAACAAAGCA
CTTCCTGCACCTATCGAGAAGACTATATCAAAAGCAAAAGGACAA
CCTAGAGAACCTCAAGTGTACACACTTCCTCCTTCAAGAGATGAA
CTTACAAAGAATCAGGTGAGTTTGACTTGCCTTGTAAAGGGCTTC
TACCCGTCAGATATCGCAGTGGAATGGGAATCAAACGGACAACCT
GAGAATAATTATAAGACTACGCCTCCTGTGCTTGATTCAGATGGA
TCATTCTTCTTGTATTCAAAGTTAACAGTTGACAAGTCTCGTTGG
CAACAAGGAAACGTGTTCAGCTGTTCAGTGATGCACGAAGCACTT
CACAACCACTACACACAGAAGTCTCTCTCACTTTCACCTGGAAAG
TGA
(SEQ ID NO: 74)
Light chain constant region of chimeric and
humanized IgG antibodies
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC
(SEQ ID NO: 65)
CGTACGGTGGCTGCACCATCTGTCTTCATCTTCCCGCCATCTGAT
GAGCAGTTGAAATCTGGAACTGCCTCTGTTGTGTGCCTGCTGAAT
AACTTCTATCCCAGAGAGGCCAAAGTACAGTGGAAGGTGGATAAC
GCCCTCCAATCGGGTAACTCCCAGGAGAGTGTCACAGAGCAGGAC
AGCAAGGACAGCACCTACAGCCTCAGCAGCACCCTGACGCTGAGC
AAAGCAGACTACGAGAAACACAAAGTCTACGCCTGCGAAGTCACC
CATCAGGGCCTGAGCTCGCCCGTCACAAAGAGCTTCAACAGGGGA
GAGTGTTAG
(SEQ ID NO: 75)
Human TROP2-Fc protein
MDMRVPAQLLGLLLLWFPGSRCHTAAQDNCTCPTNKMTVCSPDGP
GGRCQCRALGSGMAVDCSTLTSKCLLLKARMSAPKNARTLVRPSE
HALVDNDGLYDPDCDPEGRFKARQCNQTSVCWCVNSVGVRRTDKG
DLSLRCDELVRTHHILIDLRHRPTAGAFNHSDLDAELRRLFRERY
RLHPKFVAAVHYEQPTIQIELRQNTSQKAAGDVDIGDAAYYFERD
IKGESLFQGRGGLDLRVRGEPLQVERTLIYYLDEIPPKFSMKRLT
EPKSCDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSREEMTKNQVSLTCLVKGFYPSDIAVEWESNGQPENNYKTTP
PVLDSDGSFFLYSKLTVDKSRWQQGNVFSCSVMHEALHNHYTQKS
LSLSPGK
(SEQ ID NO: 66)
Human TROP2-his protein
MDMRVPAQLLGLLLLWFPGSRCHTAAQDNCTCPTNKMTVCSPDGP
GGRCQCRALGSGMAVDCSTLTSKCLLLKARMSAPKNARTLVRPSE
HALVDNDGLYDPDCDPEGRFKARQCNQTSVCWCVNSVGVRRTDKG
DLSLRCDELVRTHHILIDLRHRPTAGAFNHSDLDAELRRLFRERY
RLHPKFVAAVHYEQPTIQIELRQNTSQKAAGDVDIGDAAYYFERD
IKGESLFQGRGGLDLRVRGEPLQVERTLIYYLDEIPPKFSMKRLT
HHHHHHHHHH
(SEQ ID NO: 67)
Heavy chain of BMI (an analogue of sacituzumab)
SVQLQQSGSELKKPGASVKVSCKASGYTFTNYGMNWVKQAPGQGL
KWMGWINTYTGEPTYTDDFKGRFAFSLDTSVSTAYLQISSLKADD
TAVYFCARGGFGSSYWYFDVWGQGSLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKRVEPKSCD
KTHTCPPCPAPELLGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRE
EMTKNQVSLTCLVKGFYPSDIAVEWESNGQPENNYKTTPPVLDSD
GSFFLYSKLTVDKSRWQQGNVFSCSVMHEALHNHYTQKSLSLSPG
K
(SEQ ID NO: 68)
Light chain of BMI (an analogue of sacituzumab)
DIQLTQSPSSLSASVGDRVSITCKASQDVSIAVAWYQQKPGKAPK
LLIYSASYRYTGVPDRFSGSGSGTDFTLTISSLQPEDFAVYYCQQ
HYITPLTFGAGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
(SEQ ID NO: 69)
Cynomolgus monkey TROP2-his protein
MARGPGLAPPPLRLPLLLLLLAAVTGHTAAQDNCTCPTNKMTVCS
PDGPGGRCQCRALGSGVAVDCSTLTSKCLLLKARMSAPKNARTLV
RPNEHALVDNDGLYDPDCDPEGRFKARQCNQTSVCWCVNSVGVRR
TDKGDLSLRCDELVRTHHILIDLRHRPTAGAFNHSDLDAELRRLF
RERYRLHPKFVAAVHYEQPTIQIELRQNTSQKAAGDVDIGDAAYY
FERDVKGESLFQGRGGLDLRVRGEPLQVERTLIYYLDEIPPKFSM
KRHHHHHHHHHH
(SEQ ID NO: 70)
Full length human TROP2
MARGPGLAPPPLRLPLLLLVLAAVTGHTAAQDNCTCPTNKMTVCS
PDGPGGRCQCRALGSGMAVDCSTLTSKCLLLKARMSAPKNARTLV
RPSEHALVDNDGLYDPDCDPEGRFKARQCNQTSVCWCVNSVGVRR
TDKGDLSLRCDELVRTHHILIDLRHRPTAGAFNHSDLDAELRRLF
RERYRLHPKFVAAVHYEQPTIQIELRQNTSQKAAGDVDIGDAAYY
FERDIKGESLFQGRGGLDLRVRGEPLQVERTLIYYLDEIPPKFSM
KRLTAGLIAVIVVVVVALVAGMAVLVITNRRKSGKYKKVEIKELG
ELRKEPSLGGGGYPYDVPDYA
(SEQ ID NO: 71)
DTTP-1170 protein
MGADDVVDSSKSFVMENFSSYHGTKPGYVDSIQKGIQKPKSGTQG
NYDDDWKGFYSTDNKYDAAGYSVDNENPLSGKAGGVVKVTYPGLT
KVLALKVDNAETIKKELGLSLTEPLMEQVGTEEFIKRFGDGASRV
VLSLPFAEGSSSVEYINNWEQAKALSVELEINFETRGKRGQDAMY
EYMAQACAGNRVRRSVGSSLSCINLDWDVIRDKTKTKIESLKEHG
PIKNKMSESPNKTVSEEKAKQYLEEFHQTALEHPELSELKTVTGT
NPVFAGANYAAWAVNVAQVIDSETADNLEKTTAALSILPGIGSVM
GIADGAVHHNTEEIVAQSIALSSLMVAQAIPLVGELVDIGFAAYN
FVESIINLFQVVHNSYNRPAYSPGHKHQVQLVESGGGWVQPGGSL
RLSCAASGFTFSDTAMMWVRQAPGKGREWVAAIDTGGGYTYYADS
VKGRFTISRDNAKNTLYLQMNSLKPEDTARYYCAKTYSGNYYSNY
TVANYGTTGRGTLVTVSSHHHHHH
(SEQ ID NO: 72)
DT3C protein
MKYLLPTAAAGLLLLAAQPAMAMGADDVVDSSKSFVMENFSSYHG
TKPGYVDSIQKGIQKPKSGTQGNYDDDWKGFYSTDNKYDAAGYSV
DNENPLSGKAGGVVKVTYPGLTKVLALKVDNAETIKKELGLSLTE
PLMEQVGTEEFIKRFGDGASRVVLSLPFAEGSSSVEYINNWEQAK
ALSVELEINFETRGKRGQDAMYEYMAQACAGNRVRRSVGSSLSCI
NLDWDVIRDKTKTKIESLKEHGPIKNKMSESPNKTVSEEKAKQYL
EEFHQTALEHPELSELKTVTGTNPVFAGANYAAWAVNVAQVIDSE
TADNLEKTTAALSILPGIGSVMGIADGAVHHNTEEIVAQSIALSS
LMVAQAIPLVGELVDIGFAAYNFVESIINLFQVVHNSYNRPAYSP
GHKHIDEILAALPKTDTYKLILNGKTLKGETTTEAVDAATAEKVF
KQYANDNGVDGEWTYDDATKTFTVTEKPEVIDASELTPAVTTYKL
VINGKTLKGETTTEAVDAATAEKVFKQYANDNGVDGEWTYDDATK
TFTVTEKPEVIDASELTPAVTTYKLVINGKTLKGETTTKAVDAET
AEKAFKQYANDNGVDGVWTYDDATKTFTVTELEHHHHHH
(SEQ ID NO: 73)
Heavy chain of BM2 (an analog of Datopotamab)
QVQLVQSGAEVKKPGASVKVSCKASGYTFTTAGMQWVRQAPGQGL
EWMGWINTHSGVPKYAEDFKGRVTISADTSTSTAYLQLSSLKSED
TAVYYCARSGFGSSYWYFDVWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKRVEPKSCD
KTHTCPPCPAPELLGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRE
EMTKNQVSLTCLVKGFYPSDIAVEWESNGQPENNYKTTPPVLDSD
GSFFLYSKLTVDKSRWQQGNVFSCSVMHEALHNHYTQKSLSLSPG
K
(SEQ ID NO: 76)
Light chain of BM2 (an analog of Datopotamab)
DIQMTQSPSSLSASVGDRVTITCKASQDVSTAVAWYQQKPGKAPK
LLIYSASYRYTGVPSRFSGSGSGTDFTLTISSLQPEDFAVYYCQQ
HYITPLTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
(SEQ ID NO: 77)

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An isolated monoclonal antibody, or an antigen-binding portion thereof, capable of binding to TROP2, comprising
(i) a heavy chain variable region comprising a VH CDR1 region, a VH CDR2 region and a VH CDR3 region; and
(ii) a light chain variable region comprising a VL CDR1 region, a VL CDR2 region and a VL CDR3 region,
wherein V, CDR1 region, the VH CDR2 region and the VH CDR3 region, the VL CDR1 region, the VL CDR2 region and the VL CDR3 region comprise amino acid sequences of (1) SEQ ID NOs: 1, 2, 3, 4, 5 and 6, respectively; (2) SEQ ID NOs: 7, 8, 3, 9, 10 and 11, respectively; (3) SEQ ID NOs: 12, 13, 14, 15, 16 and 17, respectively; (4) SEQ ID NOs: 18, 19, 20, 21, 22 and 23, respectively; (5) SEQ ID NOs: 24, 25, 26, 28 and 29, respectively; (6) SEQ ID NOs: 30, 31, 32, 33, 34 and 29, respectively; or (7) SEQ ID NOs: 35, 36, 37, 36, 37, 38, 39 and 40, respectively.

2. The isolated monoclonal antibody, or the antigen-binding portion thereof, of claim 1, wherein the heavy chain variable region comprises an amino acid sequence having at least 85%, 86%, 87%, 88%, 89%, 90%, 91%, 92%, 93%, 94%, 95%, 96%, 97%, 98%, 99% or 100% identity to SEQ ID NOs: 44, 45, 46, 47, 51, 53, 55, 57, 59, or 61,
wherein the 28^{th} and 68^{th} amino acid residues in SEQ ID NO: 46 are Ser (S) and Ala (A), respectively; Thr (T) and Ala (A), respectively; or Ser (S) and Val (V), respectively,
wherein the 72^{th} and 74^{th} amino acid residues in SEQ ID NO: 47 are Arg (R) and Arg (R), respectively; or Ala (A) and Thr (T) respectively.

3. The isolated monoclonal antibody, or the antigen-binding portion thereof, of claim 1, wherein the light chain variable region comprises an amino acid sequence having at least 85%, 86%, 87%, 88%, 89%, 90%, 91%, 92%, 93%, 94%, 95%, 96%, 97%, 98%, 99% or 100% identity to SEQ ID NOs: 48, 49, 50, 52, 54, 56, 58, 60 or 62,
wherein the 1^{st}, 3^{rd}, and 78^{th} amino acid residues in SEQ ID NO: 49 are Asp (D), Leu (L), and Val (V), respectively; or Glu (E), Val (V) and Leu (L), respectively,
wherein the 42^{th}, 43^{th} and 49^{th} amino acid residues in SEQ ID NO: 50 are Gln (Q), Ser (S) and Lys (K), respectively; Gly (G), Ala (A) and Lys (K), respectively; or Gly (G), Ser (S) and Tyr (Y), respectively.

4. The isolated monoclonal antibody, or the antigen-binding portion thereof, of claim 3, wherein the heavy chain variable region and the light chain variable region comprise amino acid sequences having at least 85%, 86%, 87%, 88%, 89%, 90%, 91%, 92%, 93%, 94%, 95%, 96%, 97%, 98%, 99% or 100% identity to
(1) SEQ ID NOs: 44 and 48, respectively;
(2) SEQ ID NOs: 45 and 49, respectively, wherein the 1^{st}, 3^{rd}, and 78^{th} amino acid residues in SEQ ID NO: 49 are Asp (D), Leu (L), and Val (V), respectively;
(3) SEQ ID NOs: 46 and 49, respectively, wherein the 28^{th} and 68^{th} amino acid residues in SEQ ID NO: 46 are Ser (S) and Ala (A), respectively, wherein the 1^{st}, 3^{rd}, and 78^{th} amino acid residues in SEQ ID NO: 49 are Glu (E), Val (V) and Leu (L), respectively;
(4) SEQ ID NOs: 46 and 49, respectively, wherein the 28^{th} and 68^{th} amino acid residues in SEQ ID NO: 46 are Thr (T) and Ala (A), respectively, wherein the 1^{st}, 3^{rd}, and 78^{th} amino acid residues in SEQ ID NO: 49 are Glu (E), Val (V) and Leu (L), respectively;
(5) SEQ ID NOs: 46 and 49, respectively, wherein the 28^{th} and 68^{h} amino acid residues in SEQ ID NO: 46 are Ser (S) and Val (V), respectively, wherein the 1^{st}, 3^{rd}, and 78^{th} amino acid residues in SEQ ID NO: 49 are Glu (E), Val (V) and Leu (L), respectively;
(6) SEQ ID NOs: 47 and 49, respectively, wherein the 72^{th} and 74^{th} amino acid residues in SEQ ID NO: 47 are Arg (R) and Arg (R), respectively, wherein the 1^{st}, 3^{rd}, and 78^{th} amino acid residues in SEQ ID NO: 49 are Glu (E), Val (V) and Leu (L), respectively;
(7) SEQ ID NOs: 47 and 49, respectively, wherein the 72^{th} and 74^{th} amino acid residues in SEQ ID NO: 47 are Ala (A) and Thr (T), respectively, wherein the 1^{st}, 3^{rd}, and 78^{th} amino acid residues in SEQ ID NO: 49 are Glu (E), Val (V) and Leu (L), respectively;
(8) SEQ ID NOs: 46 and 50, respectively, wherein the 28^{th} and 68^{th} amino acid residues in SEQ ID NO: 46 are Ser (S) and Ala (A), respectively, wherein the 42^{th}, 43^{th} and 49^{th} amino acid residues in SEQ ID NO: 50 are Gln (Q), Ser (S) and Lys (K), respectively;
(9) SEQ ID NOs: 46 and 50, respectively, wherein the 28^{th} and 68^{th} amino acid residues in SEQ ID NO: 46 are Thr (T) and Ala (A), respectively, wherein the 42^{th}, 43^{th} and 49^{th} amino acid residues in SEQ ID NO: 50 are Gln (Q), Ser (S) and Lys (K), respectively;
(10) SEQ ID NOs: 46 and 50, respectively, wherein the 28^{th} and 68^{th} amino acid residues in SEQ ID NO: 46 are Ser (S) and Val (V), respectively, wherein the 42^{th}, 43^{th} and 49^{th} amino acid residues in SEQ ID NO: 50 are Gln (Q), Ser (S) and Lys (K), respectively;
(11) SEQ ID NOs: 47 and 50, respectively, wherein the 72^{th} and 74^{th} amino acid residues in SEQ ID NO: 47 are Arg (R) and Arg (R), respectively, wherein the 42^{th}, 43^{th} and 49^{th} amino acid residues in SEQ ID NO: 50 are Gln (Q), Ser (S) and Lys (K), respectively;
(12) SEQ ID NOs: 47 and 50, respectively, wherein the 72^{th} and 74^{th} amino acid residues in SEQ ID NO: 47 are Ala (A) and Thr m, respectively, wherein the 42^{th}, 43^{th} and 49^{th} amino acid residues in SEQ ID NO: 50 are Gln (Q), Ser (S) and Lys (K), respectively;
(13) SEQ ID NOs: 46 and 50, respectively, wherein the 28^{th} and 68^{th} amino acid residues in SEQ ID NO: 46 are Ser (S) and Ala (A), respectively, wherein the 42^{th}, 43^{th} and 49^{th} amino acid residues in SEQ ID NO: 50 are Gly (G), Ala (A) and Lys (K), respectively;
(14) SEQ ID NOs: 46 and 50, respectively, wherein the 28^{th} and 68^{th} amino acid residues in SEQ ID NO: 46 are Thr (T) and Ala (A), respectively, wherein the 42^{th}, 43^{th} and 49^{th} amino acid residues in SEQ ID NO: 50 are Gly (G), Ala (A) and Lys (K), respectively;
(15) SEQ ID NOs: 46 and 50, respectively, wherein the 28^{th} and 68^{th} amino acid residues in SEQ ID NO: 46 are Ser (S) and Val (V), respectively, wherein the 42^{th}, 43^{th} and 49^{th} amino acid residues in SEQ ID NO: 50 are Gly (G), Ala (A) and Lys (K), respectively;
(16) SEQ ID NOs: 47 and 50, respectively, wherein the 72^{th} and 74^{th} amino acid residues in SEQ ID NO: 47 are Arg (R) and Arg (R), respectively, wherein the 42^{th}, 43^{th} and 49^{th} amino acid residues in SEQ ID NO: 50 are Gly (G), Ala (A) and Lys (K), respectively;
(17) SEQ ID NOs: 47 and 50, respectively, wherein the 72^{th} and 74^{th} amino acid residues in SEQ ID NO: 47 are Ala (A) and Thr (T), respectively, wherein the 42^{th}, 43^{th} and 49^{th} amino acid residues in SEQ ID NO: 50 are Gly (G), Ala (A) and Lys (K), respectively;
(18) SEQ ID NOs: 46 and 50, respectively, wherein the 28^{th} and 68^{th} amino acid residues in SEQ ID NO: 46 are Ser (S) and Ala (A), respectively, wherein the 42^{th}, 43^{th} and 49^{th} amino acid residues in SEQ ID NO: 50 are Gly (G), Ser (S) and Tyr (Y), respectively;
(19) SEQ ID NOs: 46 and 50, respectively, wherein the 28^{th} and 68^{th} amino acid residues in SEQ ID NO: 46 are Thr (T) and Ala (A), respectively, wherein the 42^{th}, 43^{th} and 49^{th} amino acid residues in SEQ ID NO: 50 are Gly (G), Ser (S) and Tyr (Y), respectively;
(20) SEQ ID NOs: 46 and 50, respectively, wherein the 28^{th} and 68^{th} amino acid residues in SEQ ID NO: 46 are Ser (S) and Val (V), respectively, wherein the 42^{th}, 43^{th} and 49^{th} amino acid residues in SEQ ID NO: 50 are Gly (G), Ser (S) and Tyr (Y), respectively;
(21) SEQ ID NOs: 47 and 50, respectively, wherein the 72^{th} and 74^{th} amino acid residues in SEQ ID NO: 47 are Arg (R) and Arg (R), respectively, wherein the 42^{th}, 43^{th} and 49^{th} amino acid residues in SEQ ID NO: 50 are Gly (G), Ser (S) and Tyr (Y), respectively;
(22) SEQ ID NOs: 47 and 50, respectively, wherein the 72^{th} and 74^{th} amino acid residues in SEQ ID NO: 47 are Ala (A) and Thr (T), respectively, wherein the 42^{th}, 43^{th} and 49^{th} amino acid residues in SEQ ID NO: 50 are Gly (G), Ser (S) and Tyr (Y), respectively;
(23) SEQ ID NOs: 51 and 52, respectively;
(24) SEQ ID NOs: 53 and 54, respectively;
(25) SEQ ID NOs: 55 and 56, respectively;
(26) SEQ ID NOs: 57 and 58, respectively;
(27) SEQ ID NOs: 59 and 60, respectively; or
(28) SEQ ID NOs: 61 and 62, respectively.

5. The isolated monoclonal antibody, or the antigen-binding portion thereof, of claim 1, comprising a heavy chain constant region comprising the amino acid sequence of SEQ ID NO: 64, linked to the heavy chain variable region, and a light chain constant region comprising the amino acid sequence of SEQ ID NO: 65, linked to the light chain variable region,
wherein the 97^{th}, 239^{th} and 241^{th} amino acid residues in SEQ ID NO: 64 are Arg (R), Glu (E) and Met (M), respectively; or Lys (K), Asp (D) and Leu (L), respectively.

6. The isolated monoclonal antibody, or the antigen-binding portion thereof, of claim 1, which is an IgG1, IgG2 or IgG4 isotype.

7. The isolated monoclonal antibody, or the antigen-binding portion thereof, of claim 1, which (a) is able to bind human TROP2; (b) is able to bind monkey TROP2; and/or (c) is able to be internalized by TROP2⁺ cells.

8. The isolated monoclonal antibody, or the antigen-binding portion thereof, of claim 1, which is mouse, chimeric or humanized.

9. An immunoconjugate comprising the isolated monoclonal antibody, or the antigen-binding portion thereof, of claim 1 linked to a therapeutic agent.

10. The immunoconjugate of claim 9, wherein the therapeutic agent is a cytotoxin.

11. The immunoconjugate of claim 9, wherein the therapeutic agent is a protein comprising the amino acid sequence of SEQ ID NO: 72, or a protein comprising the amino acid sequence of SEQ ID NO: 73.

12. A nucleic acid molecule encoding the isolated monoclonal antibody, or the antigen-binding portion thereof, of claim 1.

13. An expression vector comprising the nucleic acid molecule of claim 12.

14. A host cell comprising the expression vector of claim 3.

15. A pharmaceutical composition comprising the isolated monoclonal antibody, or the antigen-binding portion thereof, of claim 1, and a pharmaceutically acceptable carrier.

16. The pharmaceutical composition of claim 15, further comprising an anti-tumor agent.

17. A method for treating a disease associated with excessive TROP2 signaling in a subject in need thereof, comprising administering to the subject a therapeutically effective amount of the pharmaceutical composition of claim 15.

18. The method of claim 17, wherein the disease is a cancer.

19. The method of claim 18, wherein the cancer is breast cancer, colorectal cancer, gastric adenocarcinoma, esophageal cancer, hepatocellular carcinoma, non-small-cell lung cancer, small-cell lung cancer, ovarian epithelial cancer, prostate cancer, pancreatic ductal adenocarcinoma, head and neck cancer, squamous cell cancer, renal cell cancer, urinary bladder neoplasm, cervical cancer, endometrial cancer, follicular thyroid cancer, or glioblastoma multiforme.

20. A method for cancer imaging in a subject in need thereof, comprising administering the subject with the isolated monoclonal antibody, or the antigen-binding portion thereof, of claim 1, wherein the isolated monoclonal antibody, or the antigen-binding portion thereof, is radioactively labeled, and the cancer is associated with excessive TROP2 signaling.


SEQUENCE LISTING (77 sequences)

SEQ ID NO: 1 - protein, 5 aa, Artificial Sequence
  VH CDR1 of mouse, chimeric and humanized A1E4F7D4
THWIH

SEQ ID NO: 2 - protein, 17 aa, Artificial Sequence
  VH CDR2 of mouse, chimeric and humanized A1E4F7D4
TIFPSNAYAVYNQKFRD

SEQ ID NO: 3 - protein, 6 aa, Artificial Sequence
  VH CDR3 of mouse, chimeric and humanized A1E4F7D4, and mouse A1E11A12D1
ASYFDY

SEQ ID NO: 4 - protein, 11 aa, Artificial Sequence
  VL CDR1 of mouse, chimeric and humanized A1E4F7D4
RASQNIGTSIH

SEQ ID NO: 5 - protein, 7 aa, Artificial Sequence
  VL CDR2 of mouse, chimeric and humanized A1E4F7D4
YASESIS

SEQ ID NO: 6 - protein, 9 aa, Artificial Sequence
  VL CDR3 of mouse, chimeric and humanized A1E4F7D4
QHSHSWPFT

SEQ ID NO: 7 - protein, 5 aa, Artificial Sequence
  VH CDR1 of mouse A1E11A12D1
TYGIS

SEQ ID NO: 8 - protein, 17 aa, Artificial Sequence
  VH CDR2 of mouse A1E11A12D1
QIYPGSDYSYCDEDFKG

SEQ ID NO: 9 - protein, 11 aa, Artificial Sequence
  VL CDR1 of mouse A1E11A12D1
RASQTIGTAIH

SEQ ID NO: 10 - protein, 7 aa, Artificial Sequence
  VL CDR2 of mouse A1E11A12D1
YASESIS

SEQ ID NO: 11 - protein, 9 aa, Artificial Sequence
  VL CDR3 of mouse A1E11A12D1
QQSNNWPFT

SEQ ID NO: 12 - protein, 5 aa, Artificial Sequence
  VH CDR1 of mouse A1H3C5H8E12
NYWID

SEQ ID NO: 13 - protein, 17 aa, Artificial Sequence
  VH CDR2 of mouse A1H3C5H8E12
NIFPGGFYTNYNEKFKG

SEQ ID NO: 14 - protein, 6 aa, Artificial Sequence
  VH CDR3 of mouse A1H3C5H8E12
GGVFDY

SEQ ID NO: 15 - protein, 11 aa, Artificial Sequence
  VL CDR1 of mouse A1H3C5H8E12
RASQSIGTSIH

SEQ ID NO: 16 - protein, 7 aa, Artificial Sequence
  VL CDR2 of mouse A1H3C5H8E12
YASESIS

SEQ ID NO: 17 - protein, 9 aa, Artificial Sequence
  VL CDR3 of mouse A1H3C5H8E12
QQSNSWPYT

SEQ ID NO: 18 - protein, 5 aa, Artificial Sequence
  VH CDR1 of mouse and chimeric A1F1G12A7
SYGVH

SEQ ID NO: 19 - protein, 16 aa, Artificial Sequence
  VH CDR2 of mouse and chimeric A1F1G12A7
VIWRGGITDYNAAFIS

SEQ ID NO: 20 - protein, 11 aa, Artificial Sequence
  VH CDR3 of mouse and chimeric A1F1G12A7
DGDYEYYTMDY

SEQ ID NO: 21 - protein, 15 aa, Artificial Sequence
  VL CDR1 of mouse and chimeric A1F1G12A7
RASKSVSTSGYSYMH

SEQ ID NO: 22 - protein, 7 aa, Artificial Sequence
  VL CDR2 of mouse and chimeric A1F1G12A7
LASDQDS

SEQ ID NO: 23 - protein, 9 aa, Artificial Sequence
  VL CDR3 of mouse and chimeric A1F1G12A7
QHSRELPYT

SEQ ID NO: 24 - protein, 5 aa, Artificial Sequence
  VH CDR1 of mouse A1B12D2B4E7B3
DYYMN

SEQ ID NO: 25 - protein, 17 aa, Artificial Sequence
  VH CDR2 of mouse A1B12D2B4E7B3
YIYPNHGGTGYNQKFKD

SEQ ID NO: 26 - protein, 9 aa, Artificial Sequence
  VH CDR3 of mouse A1B12D2B4E7B3
ENYGYAMDY

SEQ ID NO: 27 - protein, 16 aa, Artificial Sequence
  VL CDR1 of mouse A1B12D2B4E7B3
RSSQSLVHGDGNTYLH

SEQ ID NO: 28 - protein, 7 aa, Artificial Sequence
  VL CDR2 of mouse A1B12D2B4E7B3
TVSNRFS

SEQ ID NO: 29 - protein, 8 aa, Artificial Sequence
  VL CDR3 of mouse A1B12D2B4E7B3 and mouse B1G1F5A3
SQTTHVPT

SEQ ID NO: 30 - protein, 5 aa, Artificial Sequence
  VH CDR1 of mouse B1G1F5A3
DYSMN

SEQ ID NO: 31 - protein, 17 aa, Artificial Sequence
  VH CDR2 of mouse B1G1F5A3
YIYPNNGASGFNQKFKG

SEQ ID NO: 32 - protein, 11 aa, Artificial Sequence
  VH CDR3 of mouse B1G1F5A3
EQDNSGYCFDY

SEQ ID NO: 33 - protein, 16 aa, Artificial Sequence
  VL CDR1 of mouse B1G1F5A3
RSSLNLVHSNGNTFLH

SEQ ID NO: 34 - protein, 7 aa, Artificial Sequence
  VL CDR2 of mouse B1G1F5A3
KVSNRFS

SEQ ID NO: 35 - protein, 5 aa, Artificial Sequence
  VH CDR1 of mouse and chimeric C1B3B12D2
KFWIG

SEQ ID NO: 36 - protein, 17 aa, Artificial Sequence
  VH CDR2 of mouse and chimeric C1B3B12D2
NIYPGGAYINYNENFKG

SEQ ID NO: 37 - protein, 6 aa, Artificial Sequence
  VH CDR3 of mouse and chimeric C1B3B12D2
EGSSGY

SEQ ID NO: 38 - protein, 17 aa, Artificial Sequence
  VL CDR1 of mouse and chimeric C1B3B12D2
KCSQSLLNSGTQENYLT

SEQ ID NO: 39 - protein, 7 aa, Artificial Sequence
  VL CDR2 of mouse and chimeric C1B3B12D2
WASIREP

SEQ ID NO: 40 - protein, 9 aa, Artificial Sequence
  VL CDR3 of mouse and chimeric C1B3B12D2
QHDYSYPFT

SEQ ID NO: 41 - DNA, 345 bp, Artificial Sequence
  VH of mouse and chimeric A1E4F7D4
caggtccagctgcagcagtctggggctgagctggcaaaacctgggacctcagtgaagatgtcctgcgaggcttctggctactcctttactacccactggatacactggatgaagcagaggcctggacagggtctggaatggattgggactatttttcctagcaatgcttatgctgtttataatcagaaattcagggacagggccataatgactgcagacagatcctccactacagcctatatacaactcaccggcctgacatctgaggactctgcagtctattactgtgcaagagccagttactttgactactggggccaaggcaccactctcacagtctcctca

SEQ ID NO: 42 - DNA, 345 bp, Artificial Sequence
  VH of huA1E4F7D4-V6, huA1E4F7D4-V11, huA1E4F7D4-V16 and huA1E4F7D4-V21
caagtgcagctggtgcagtctggggctgaggtgaagaagcctggggcctctgtgaaggtgagctgcaaggcctctggctacagcttcaccacccactggatccactgggtgagacaagcccctggccaaggcctggagtggattggcaccatcttccctagcaatgcctatgctgtgtacaatcagaagttcagagacagagccaccatgacagctgacacaagcatcagcacagcctacattgagctgagcagactgagatctgatgacacagctgtgtactactgtgctagagctagctactttgactactggggccaaggcaccacagtgacagtgagcagc

SEQ ID NO: 43 - DNA, 321 bp, Artificial Sequence
  VL of mouse and chimeric A1E4F7D4
gacatcttgttgactcagtctccagccaccctgtctgtgagtccaggagaaagagtcactttctcctgcagggccagtcagaacattggcacaagcatacactggtttcagcaaagaacaactggttctccaaggcttctcataaagtatgcttctgagtctatctctgggatcccttccagatttagtggcagtggatcagggacagattttactcttcacatcaacagtgtggagtctgaagatattgcacattattactgtcaacatagtcatagctggccattcacgttcggctcggggacaaagttggaaatacaa

SEQ ID NO: 44 - protein, 115 aa, Artificial Sequence
  VH of mouse and chimeric A1E4F7D4
QVQLQQSGAELAKPGTSVKMSCEASGYSFTTHWIHWMKQRPGQGLEWIGTIFPSNAYAVYNQKFRDRAIMTADRSSTTAYIQLTGLTSEDSAVYYCARASYFDYWGQGTTLTVSS

SEQ ID NO: 45 - protein, 115 aa, Artificial Sequence
  VH of huA1E4F7D4-V1
QVQLVQSGAEVKKPGASVKVSCKASGYSFTTHWIHWMKQRPGQGLEWIGTIFPSNAYAVYNQKFRDRAIMTADRSISTAYIELTRLRSDDTAVYYCARASYFDYWGQGTTVTVSS

SEQ ID NO: 46 - protein, 115 aa, Artificial Sequence
  VH of huA1E4F7D4-V2-huA1E4F7D4-V4, huA1E4F7D4-V7-huA1E4F7D4-V9, huA1E4F7D4-V12-huA1E4F7D4-V14, and huA2G10B1C2-V17-huA1E4F7D4-V19
  misc_feature (28)..(28): Xaa can be Ser or Thr
  misc_feature (68)..(68): Xaa can be Ala or Val
QVQLVQSGAEVKKPGASVKVSCKASGYXFTTHWIHWVRQAPGQGLEWIGTIFPSNAYAVYNQKFRDRXTMTADRSISTAYIELSRLRSDDTAVYYCARASYFDYWGQGTTVTVSS

SEQ ID NO: 47 - protein, 115 aa, Artificial Sequence
  VH of huA1E4F7D4-V5, huA1E4F7D4-V6, huA1E4F7D4-V10, huA1E4F7D4-V11, huA1E4F7D4-V15, huA1E4F7D4-V16, huA1E4F7D4-V20 and huA1E4F7D4-V21
  misc_feature (72)..(72): Xaa can be Arg or Ala
  misc_feature (74)..(74): Xaa can be Arg or Thr
QVQLVQSGAEVKKPGASVKVSCKASGYSFTTHWIHWVRQAPGQGLEWIGTIFPSNAYAVYNQKFRDRATMTXDXSISTAYIELSRLRSDDTAVYYCARASYFDYWGQGTTVTVSS

SEQ ID NO: 48 - protein, 107 aa, Artificial Sequence
  VL of mouse and chimeric A1E4F7D4
DILLTQSPATLSVSPGERVTFSCRASQNIGTSIHWFQQRTTGSPRLLIKYASESISGIPSRFSGSGSGTDFTLHINSVESEDIAHYYCQHSHSWPFTFGSGTKLEIQ

SEQ ID NO: 49 - protein, 107 aa, Artificial Sequence
  VL of huA1E4F7D4-V1-huA1E4F7D4-V6
  misc_feature (1)..(1): Xaa can be Asp or Glu
  misc_feature (3)..(3): Xaa can be Ile or Val
  misc_feature (78)..(78): Xaa can be Val or Leu
XIXLTQSPATLSLSPGERATLSCRASQNIGTSIHWFQQKPGGSPRLLIKYASESISGIPSRFSGSGSGTDFTLTISSXEPEDFAVYYCQHSHSWPFTFGQGTKLEIK

SEQ ID NO: 50 - protein, 107 aa, Artificial Sequence
  VL of huA1E4F7D4-V7 - huA1E4F7D4-V21
  misc_feature (42)..(42): Xaa can be Gln or Gly
  misc_feature (43)..(43): Xaa can be Ser or Ala
  misc_feature (49)..(49): Xaa can be Lys or Tyr
EIVLTQSPATLSLSPGERATLSCRASQNIGTSIHWFQQKPGXXPRLLIXYASESISGIPSRFSGSGSGTDFTLTISSLEPEDFAVYYCQHSHSWPFTFGQGTKLEIK

SEQ ID NO: 51 - protein, 115 aa, Artificial Sequence
  VH of mouse A1E11A12D1
QVQLQQSGAELARPGASVKLSCMASGYTFTTYGISWVRQRTGQGLEWIGQIYPGSDYSYCDEDFKGKATLTADKSSSTAYMQLSSLTSEDSAVYFCARASYFDYWGQGTTLTVSS

SEQ ID NO: 52 - protein, 107 aa, Artificial Sequence
  VL of mouse A1E11A12D1
DILLTQSPAILSVSPGERVSFSCRASQTIGTAIHWYQQRANGSPRLLIKYASESISGIPSRFSGSGSGTDFALSINTVESEDFAYYYCQQSNNWPFTFGGGTKLEIR

SEQ ID NO: 53 - protein, 115 aa, Artificial Sequence
  VH of mouse A1H3C5H8E12
QVQLQQSGAELVRPGTSVKMSCKAAGYTFTNYWIDWVKQRPGHGLEWIGNIFPGGFYTNYNEKFKGKATLTADTSSSTAYMQLSSLTSEDSAIYYCARGGVFDYWGQGTTLTVSS

SEQ ID NO: 54 - protein, 107 aa, Artificial Sequence
  VL of mouse A1H3C5H8E12
DILLTQSPAILSVSPGERVSFSCRASQSIGTSIHWYQQRTNGSPRLLIKYASESISGIPSRFSGSGSGTDFTLSINSVESEDIADYYCQQSNSWPYTFGGGTKLEIK

SEQ ID NO: 55 - protein, 119 aa, Artificial Sequence
  VH of mouse and chimeric A1F1G12A7
QVQLKQSGPGLVQPSQSLSITCTVSGFSLTSYGVHWVRQSPGKGLEWLGVIWRGGITDYNAAFISRLSISKDNSKSQIFFKMNSLQGDDTAIYYCARDGDYEYYTMDYWGQGTSVTVSS

SEQ ID NO: 56 - protein, 111 aa, Artificial Sequence
  VL of mouse and chimeric A1F1G12A7
DIVLTQSPASLAVSLGQRATISCRASKSVSTSGYSYMHWNQQKPGQSPKLLIYLASDQDSGVPARFSGSGSGTDFTLSIHPVEEEDAATYYCQHSRELPYTFGGGTKLEIK

SEQ ID NO: 57 - protein, 118 aa, Artificial Sequence
  VH of mouse A1B12D2B4E7B3
EVQLQQSGPELVKPGASVKMSCKASGFTFTDYYMNWVKQSHGKSLEWIGYIYPNHGGTGYNQKFKDKATLTVDKSSSTAYMELRSLTSDDSAVYYCARENYGYAMDYWGQGTSVTVSS

SEQ ID NO: 58 - protein, 111 aa, Artificial Sequence
  VL of mouse A1B12D2B4E7B3
DVVMTQTPLSLPVSLGDQASISCRSSQSLVHGDGNTYLHWFLQKPGQSPKLLIYTVSNRFSGVPDRFSGSGSGTDFTLKINRVEAEDLGIYFCSQTTHVPTFGGGTKLEIK

SEQ ID NO: 59 - protein, 120 aa, Artificial Sequence
  VH of mouse B1G1F5A3
EVQLQQSGPELVKPGTSVKMSCKASGYSFADYSMNWVRQSHGNSLEWIGYIYPNNGASGFNQKFKGKATLTVDKSSSTAYMELHSLTSEDSAVYYCAREQDNSGYCFDYWGQGTSLTVSS

SEQ ID NO: 60 - protein, 111 aa, Artificial Sequence
  VL of mouse B1G1F5A3
DVVMTQTPLSLAVRLGDQASISCRSSLNLVHSNGNTFLHWYLQRPGQSPKLLIYKVSNRFSGVSDRFSGSGSGTDFTLKISRVEAEDLGVYFCSQTTHVPTFGGGTKLEIK

SEQ ID NO: 61 - protein, 115 aa, Artificial Sequence
  VH of mouse and chimeric C1B3B12D2
QVQLQQSGPELVRPGTSVKMSCKAAGYTFTKFWIGWVKQRPGHGLEWIGNIYPGGAYINYNENFKGKATLTADTFSSTAYMQLNSLTSADSAIYYCAREGSSGYWGQGTTLTVSS

SEQ ID NO: 62 - protein, 113 aa, Artificial Sequence
  VL of mouse and chimeric C1B3B12D2
DIVMTQSPSSLTVTAGEKVTLSCKCSQSLLNSGTQENYLTWYQQKPGQPPKMLIYWASIREPGVPDRFTGSGSGTDFTLTINNVQAEDLAVYYCQHDYSYPFTFGSGTKLEIK

SEQ ID NO: 63 - DNA, 321 bp, Artificial Sequence
  VL of huA1E4F7D4-V12 - huA1E4F7D4-V16
gagattgtgctgacacagagccctgccaccctgagcctgtcccctggggagagagctaccctgagctgcagagcttctcagaacattggcacaagcatccactggtttcagcagaagcctgggggcgcccctagactgctgatcaagtatgcctctgagagcatctctggcatccctagcagattctctggctctggctctggcacagacttcaccctgaccatcagcagcctggagcctgaggactttgctgtgtactactgtcagcacagccacagctggcccttcacctttggccaaggcaccaagctggagatcaag

SEQ ID NO: 64 - protein, 330 aa, Artificial Sequence
  Heavy chain constant region of chimeric and humanized IgG antibodies
  misc_feature (97)..(97): Xaa can be Arg or Lys
  misc_feature (239)..(239): Xaa can be Glu or Asp
  misc_feature (241)..(241): Xaa can be Met or Leu
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 107 aa, Artificial Sequence [sequence omitted]

SEQ ID NO: 66 - protein, 502 aa, Artificial Sequence
  Human TROP2-Fc protein
MDMRVPAQLLGLLLLWFPGSRCHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 280 aa, Artificial Sequence
  Human TROP2-his protein
MDMRVPAQLLGLLLLWFPGSRCHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTHHHHHHHHHH

SEQ ID NO: 68 - protein, 451 aa, Artificial Sequence
  Heavy chain of an analogue of sacituzumab
SVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 214 aa, Artificial Sequence
  Light chain of an analogue of sacituzumab
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 282 aa, Artificial Sequence
  Cynomolgus monkey TROP2-his protein
MARGPGLAPPPLRLPLLLLLLAAVTGHTAAQDNCTCPTNKMTVCSPDGPGGRCQCRALGSGVAVDCSTLTSKCLLLKARMSAPKNARTLVRPNEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDVKGESLFQGRGGLDLRVRGEPLQVERTLIYYLDEIPPKFSMKRHHHHHHHHHH

SEQ ID NO: 71 - protein, 336 aa, Artificial Sequence
  Full length human TROP2
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSLGGGGYPYDVPDYA

SEQ ID NO: 72 - protein, 519 aa, Artificial Sequence
  DTTP-1170 protein
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKHQVQLVESGGGWVQPGGSLRLSCAASGFTFSDTAMMWVRQAPGKGREWVAAIDTGGGYTYYADSVKGRFTISRDNAKNTLYLQMNSLKPEDTARYYCAKTYSGNYYSNYTVANYGTTGRGTLVTVSSHHHHHH

SEQ ID NO: 73 - protein, 624 aa, Artificial Sequence
  DT3C protein
MKYLLPTAAAGLLLLAAQPAMAMGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKHIDEILAALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTELEHHHHHH

SEQ ID NO: 74 - DNA, 993 bp, Artificial Sequence
  Heavy chain constant region of chimeric and humanized IgG antibodies
gcctctactaaagggccctcggtattcccgctggcaccttcatcaaagtccacttcaggaggaacagcagcacttggatgtctcgttaaggactatttcccagaaccagtcactgtttcctggaattcaggagcacttacatcaggagtgcacacatttcctgcagtgcttcaatcatcaggactttactcactatcctcggtagtcacggtgccttcatcatcacttggaacacaaacatacatctgcaacgtgaaccacaaaccttcgaatactaaagtcgataagaaggtcgagcctaaatcatgcgataagacccatacatgccctccttgccctgcacctgaacttcttggagggccgagtgtgtttctgtttcctcctaagcccaaggatacacttatgatctcaagaacacctgaagtgacatgcgtggtggtggatgtgtcacacgaagatcctgaagtgaaatttaactggtacgtggatggagtggaagtgcacaacgcaaagactaagcctagagaagaacaatacaactcaacatacagagtggtgtcagtgcttacagtgcttcaccaagattggcttaacggaaaggagtataaatgcaaagtgtcaaacaaagcacttcctgcacctatcgagaagactatatcaaaagcaaaaggacaacctagagaacctcaagtgtacacacttcctccttcaagagatgaacttacaaagaatcaggtgagtttgacttgccttgtaaagggcttctacccgtcagatatcgcagtggaatgggaatcaaacggacaacctgagaataattataagactacgcctcctgtgcttgattcagatggatcattcttcttgtattcaaagttaacagttgacaagtctcgttggcaacaaggaaacgtgttcagctgttcagtgatgcacgaagcacttcacaaccactacacacagaagtctctctcactttcacctggaaagtga

SEQ ID NO: 75 - DNA, 324 bp, Artificial Sequence
  Light chain constant region of chimeric and humanized IgG antibodies
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 76 - protein, 451 aa, Artificial Sequence
  Heavy chain of BM2
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 214 aa, Artificial Sequence
  Light chain of BM2
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC